(19) United States
(12) Patent Application Publication
(10) Pub. No.: US 2003/0170246 A1
(43) Pub. Date: Sep. 11, 2003

(54) Lipophilin complexes for use in cancer diagnosis and therapy

(75) Inventors: Gary R. Fanger, Mill Creek, WA (US); Margarita Durham, Seattle, WA (US); Raymond L. Houghton, Bothell, WA (US); Davin C. Dillon, Issaquah, WA (US); Darrick Carter, Seattle, WA (US); David H. Persing, Redmond, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 10/096,319

(22) Filed: Mar. 12, 2002

Related U.S. Application Data
(63) Continuation-in-part of application No. 09/905,673, filed on Jul. 13, 2001.
Application No. 09/905,673 is a continuation-in-part of application No. 09/780,842, filed on Feb. 8, 2001, now abandoned.
(60) Provisional application No. 60/215,735, filed on Jun. 28, 2000.
(60) Provisional application No. 60/183,495, filed on Feb. 11, 2000.

(51) Int. Cl. A61K 39/395; C07H 21/04; C12P 21/04; C12N 5/06; C07K 14/435

(52) U.S. Cl. 424/155.1; 530/350; 435/69.7; 435/320.1; 435/325; 536/23.5; 530/388.1

(57) ABSTRACT

Compositions and methods for the therapy and diagnosis of breast, ovarian and prostate cancer are disclosed. Compositions may comprise one or more lipophilin fusion proteins, which comprise at least two different lipophilin-like polypeptides linked by a peptide bond. Such compositions may be used for the prevention and treatment of breast, ovarian and prostate cancer. Diagnostic methods based on detecting the presence of lipophilin complexes, or antibodies thereto, in a patient are also provided.

### STATEMENT REGARDING FEDERALLY SPONSORED RESEARCH OR DEVELOPMENT

This invention was made in part with government support under NCI grant numbers CA-75794 and CA-86673. The Government may have certain rights in this invention.

### BACKGROUND OF THE INVENTION

1. Field of the Invention

The present invention relates generally to therapy and diagnosis of cancer, such as breast, prostate and ovarian cancer. The invention is more specifically related to the use of lipophilin complexes for prevention and treatment Of such cancers, as well as for cancer diagnosis and monitoring.

2. Description of the Related Art

Cancer is a significant health problem throughout the world. Although advances have been made in detection and treatment of this disease, breast cancer remains the second leading cause of cancer-related deaths in women, affecting more than 180,000 women in the United States each year. For women in North America, the lifetime odds of getting breast cancer are now one in eight.

No vaccine or other universally successful method for the prevention or treatment of breast and other cancers is currently available. Management of these diseases currently relies on a combination of early diagnosis (through routine screening procedures) and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. See, e.g., Porter-Jordan and Lippman, Breast Cancer 8:73-100, 1994. However, the use of established markers often leads to a result that is difficult to interpret, and the high mortality observed for many cancer types indicates that improvements are needed in the treatment, diagnosis and prevention of the disease.

In order to improve cancer treatment and survival, it would be beneficial to identify markers that permit an earlier and/or more accurate diagnosis and that may facilitate the selection of a course of treatment and monitoring of patients. Such markers may further form the basis for more effective therapies for certain cancers. For example, immunotherapies employing such proteins, or the DNA encoding the proteins, have the potential to substantially improve cancer treatment and survival. Such therapies may involve the generation or enhancement of an immune response to an antigenic tumor marker.

Accordingly, there is a need in the art for cancer antigens and markers, and for improved diagnostic methods and therapies for these cancers. The present invention fulfills these needs and further provides other related advantages.

### BRIEF SUMMARY OF THE INVENTION

Briefly stated, the present invention provides compositions and methods for the diagnosis and therapy of cancers, such as breast, ovarian and prostate cancers. Within certain aspects, the present invention provides isolated lipophilin fusion proteins, as well as DNA molecules and constructs encoding such fusion proteins, comprising a first lipophilin-like polypeptide linked to a second lipophilin-like polypeptide or a breast tumor antigen by a peptide bond. In specific embodiments, the first lipophilin-like polypeptide is a mammaglobin or mammaglobin-like polypeptide and/or the second lipophilin-like polypeptide is a lipophilin B or lipophilin B-like polypeptide, and the breast tumor antigen is B305D.

In further aspects, the present invention provides pharmaceutical compositions comprising a fusion protein, or a polynucleotide encoding the fusion protein as described above, in combination with a physiologically acceptable carrier.

Within further aspects, the present invention provides antibodies, such as monoclonal antibodies, or antigen-binding fragments thereof, that bind to a fusion protein as described above, wherein the antibodies or fragments thereof do not detectably bind to mammaglobin, lipophilin, e.g., lipophilin A, lipophilin B or lipophilin C, or to B305D.

The present invention further provides pharmaceutical compositions that comprise: (a) an antibody or antigen-binding fragment thereof that specifically binds to a complex as described above; and (b) a physiologically acceptable carrier.

Within further aspects, the present invention provides methods for inhibiting the development of cancer, such as breast, ovarian or prostate cancer, in a patient, comprising administering to a patient a pharmaceutical composition or vaccine as recited above. The patient may be afflicted with a cancer, in which case the methods provide treatment for the disease, or a patient considered at risk for such a disease may be treated prophylactically.

The present invention further provides, within other aspects, methods for removing tumor cells from a biological sample, comprising contacting a biological sample with T cells that specifically react with lipophilin complex, wherein the step of contacting is performed under conditions and for a time sufficient to permit the removal of cells expressing the complex from the sample.

Within related aspects, methods are provided for inhibiting the development of a cancer in a patient, comprising administering to a patient a biological sample treated as described above.

Methods are further provided, within other aspects, for stimulating and/or expanding T cells specific for a lipophilin complex, comprising contacting T cells with one or more of: (i) a lipophilin complex as described above; (ii) a polynucleotide encoding such a complex; and/or (iii) an antigen presenting cell that expresses such a complex; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of a lipophilin complex; (ii) a polynucleotide encoding such a complex; and (iii) an antigen-presenting cell that expressed such a complex; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer in a patient, comprising (a) contacting a biological sample obtained from a patient with an antibody or antigen-binding fragment thereof that specifically binds to a complex as described above; (b) detecting in the sample an amount of complex that binds to the antibody or fragment thereof; and (c) comparing the amount of complex with a predetermined cut-off value. Within preferred embodiments, the antibody is a monoclonal antibody. Step (b) may comprise, for example, a two-antibody sandwich assay. In certain embodiments, the level of free components of the complex may also be assayed, and the ratio of complex to components determined. In such embodiments, step (c) involves comparing the ratio to a predetermined cut-off value.

The present invention also provides, within other aspects, methods for monitoring the progression of a cancer in a patient. Such methods comprise the steps of: (a) contacting a biological sample obtained from a patient at a first point in time with an antibody or antigen-binding fragment thereof that specifically binds to a complex as described above; (b) detecting in the sample an amount of complex that binds to the antibody or fragment thereof; (c) repeating steps (a) and (b) using a biological sample obtained from the patient at a subsequent point in time; and (d) comparing the amount of complex detected in step (c) with the amount detected in step (b). In certain embodiments, the level of free components of the complex may also be assayed, and the change in the ratio of complex to components compared in step (d).

Within further aspects, the present invention provides antibodies, such as monoclonal antibodies, that bind to a polypeptide complex as described above, as well as diagnostic kits comprising such antibodies. Diagnostic kits comprising one or more oligonucleotide probes or primers as described above are also provided.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIG. 1 is a diagram depicting a lipophilin complex comprising glycosylated mammaglobin and lipophilin B. Disulfide bonds link the two component proteins, which are released upon reduction of the complex.

FIGS. 2A and 2B are photographs illustrating the association of mammaglobin and lipophilin B by disulfide bonds. FIG. 2A depicts purified mammaglobin analyzed on a 4-20% SDS-PAGE gel stained with Glyco-Pro Glycoprotein detection kit (Sigma Chemical Co., St. Louis, Mo.). Lane 1 shows the complex in non-reducing SDS sample buffer, and lane 2 shows the complex with 10 mM DTT in the sample buffer. FIG. 2B depicts the same gel restained with silver stain. The arrow denotes the weakly staining mammaglobin. A new band is detected in the reduced lane (lane 2). The position of molecular weight markers is indicated on the left side of each figure.

FIGS. 3A and 3B illustrate the results of N-terminal sequencing of a lipophilin identified as being associated with mammaglobin. The first ten N-terminal residues were determined, as indicated.

FIGS. 4A, 4B, and 4C show that mammaglobin is linked to lipophilin B via cysteine bridge from mammaglobin amino acid 4 to lipophilin B amino acid 67. The 1621.6 ion matches the combined molecular weight of mammaglobin tryptic fragment 1-13 and lipophilin B tryptic fragment 67-69. Upon reduction, mammaglobin tryptic fragment 1-13 (MW 1316.5) and lipophilin B tryptic fragment 67-69 (MW 308.1) are visible.

FIGS. 5A and 5B show a titration curve for a mammaglobin capture ELISA using the R062/R028-biotin antibodies. The antibodies were tested on HEK293-wild type supernatants (negative control), HEK293 transfected with mammaglobin (negative control), HEK293 transfected with mammaglobin-lipophilin B (positive control), and MB415 supernatants (positive control).

FIGS. 6A and 6B show a titration curve for a mammaglobin capture ELISA using the the RO48/RO28-biotin antibodies. The antibodies were tested on the same samples as described in FIG. 5.

FIGS. 7A and 7B show a titration curve for a mammaglobin capture ELISA using the the RO28/RO48-biotin antibodies. The antibodies were tested on the same samples as described in FIG. 5.

### DETAILED DESCRIPTION OF THE INVENTION

U.S. patent application publications, U.S. patent applications, foreign patents, foreign patent applications and non-patent publications referred to in this specification and/or listed in the Application Data Sheet, are incorporated herein by reference, in their entirety.

As noted above, the present invention is generally directed to compositions and methods for the therapy and diagnosis of cancer, such as breast, ovarian and prostate cancer. The compositions described herein generally include one or more lipophilin complexes (i.e., complexes comprising two different lipophilin-like polypeptides, linked by one or more disulfide bonds). Preferably one lipophilin-like polypeptide is a mammaglobin or a mammaglobin-like polypeptide; more preferably the complex comprises a mammaglobin polypeptide and a lipophilin B polypeptide. The present invention is based, in part, on the discovery that such complexes form in breast tumor cells, and may be of diagnostic, prognostic and therapeutic use. The invention further provides antibodies, and antigen-binding fragments thereof, that specifically bind to lipophilin complexes. Such antibodies may be used within therapeutic and diagnostic methods, as described herein.

Lipophilin Complexes

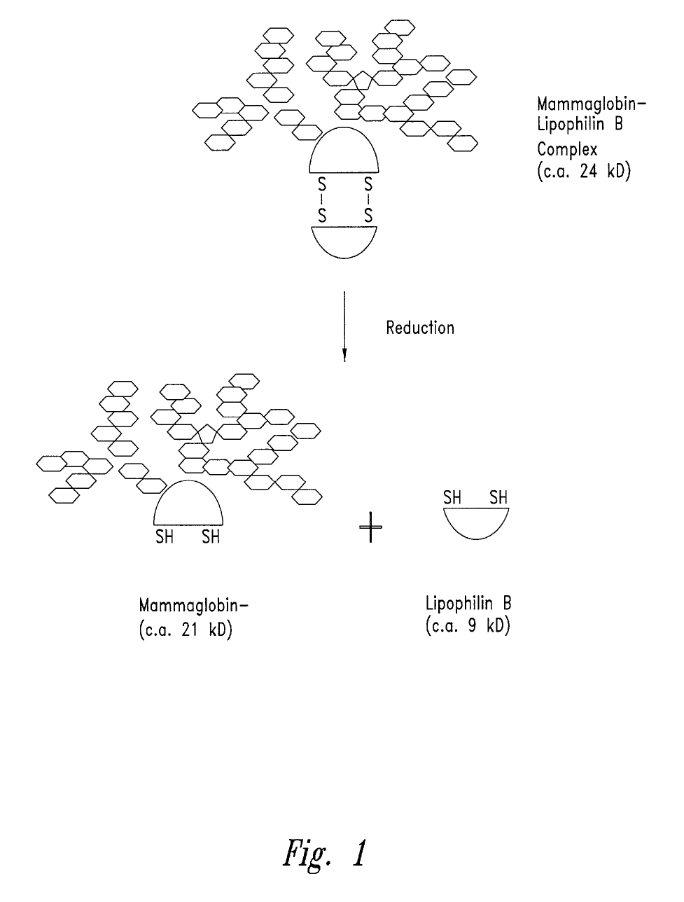

A lipophilin complex is an association of at least two different lipophilin-like polypeptides linked by disulfide bonds. Lipophilin-like proteins are members of the uteroglobin superfamily of proteins, and include mammaglobin (SEQ ID NO:1), as well as lipophilin A, lipophilin B (SEQ ID NO:2) and lipophilin C (also known as mammaglobin B) (see Zhao et al., Biochem. Biophys. Res. Comm. 256:147-155,1999; Lehrer et al., FEBS Letters 432:163-167,1998). Preferred complexes comprise a mammaglobin polypeptide, most preferably associated with (e.g., linked via disulfide bonds to) a lipophilin B polypeptide. The mammaglobin polypeptide within such complexes is preferably glycosylated (as indicated in FIG. 1).

Within the context of the present invention, two lipophilin-like polypeptides are said to form a complex if the polypeptides are linked. The linkage between the two lipophilin-like polypeptides may vary, but will typically comprise one or more covalent linkages, and most typically will comprise one or more disulfide bonds. Such disulfide linkages may be detected, for example, based on the presence of a higher molecular weight complex under non-reducing conditions, and the separation of the complex into lower molecular weight components under reducing conditions (e.g., using SDS-PAGE analysis as described herein). In general, such a complex should not detectably dissociate in the presence of chaotropic agents (e.g., 8 M urea, 6 M guanidine hydrochloride or boiling in SDS). The use of such agents is well known in the art.

A lipophilin-like polypeptide is a polypeptide that comprises: (i) a native lipophilin or mammaglobin protein, (ii) a portion of such a protein that is capable of forming a complex as described above, or (iii) a variant of such a protein that differs in one or more substitutions, deletions, additions and/or insertions, such that the ability of the variant to form a lipophilin complex is not substantially diminished. In other words, the ability of a portion or other variant to associate with a lipophilin-like partner may be enhanced or unchanged, relative to the native lipophilin-like protein, or may be diminished by less than 50%, and preferably less than 20%, relative to the native lipophilin-like protein. For example, a mammaglobin polypeptide may comprise a full length native mammaglobin sequence, or a portion or other variant of such a sequence, provided that the ability of the polypeptide to associate with at least one other lipophilin-like protein (preferably lipophilin B) is not diminished, relative to the ability of a native mammaglobin. Such a polypeptide is preferably glycosylated. Similarly, a lipophilin B polypeptide may comprise a full length native lipophilin B molecule, or a portion or other variant thereof that associates with at least one other lipophilin-like protein (preferably mammaglobin) to form a complex as described above.

Lipophilin-like protein variants may generally be identified by modifying a lipophilin-like protein sequence and evaluating the ability to form a complex. Preferred variants include those in which substitutions are made at no more than 20% of the residues in the native sequence.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. Variants may also (or alternatively) be modified by, for example, the deletion or addition of amino acids that have minimal influence on the immunogenicity, secondary structure and hydropathic nature of the polypeptide.

Polypeptides may further comprise sequences not normally present within a native lipophilin. Such sequences include signal (or leader) sequences at the N-terminal end of the polypeptide, which co-translationally or post-translationally direct transfer of the polypeptide. A polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides may be prepared using any of a variety of well known techniques. Recombinant polypeptides may be readily prepared from DNA sequences using any of a variety of expression vectors known to those of ordinary skill in the art. Expression may be achieved in any appropriate host cell that has been transformed or transfected with an expression vector containing a DNA molecule that encodes a recombinant polypeptide Suitable host cells include prokaryotes, yeast and higher eukaryotic cells, such as mammalian and plant cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

In order to express a desired polypeptide, the nucleotide sequences encoding the polypeptide, or functional equivalents, may be inserted into appropriate expression vector, i.e., a vector that contains the necessary elements for the transcription and translation of the inserted coding sequence. Methods that are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The expression “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

Polypeptides having fewer than about 150 amino acids may also be generated by synthetic means using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc. (Foster City, Calif.), and may be operated according to the manufacturer's instructions.

In general, lipophilin-like polypeptides as described herein are isolated. An “isolated” polypeptide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure.

The present invention further provides, in other aspects, fusion proteins that comprise at least one lipophilin polypeptide as described above, as well as polynucleotides encoding such fusion proteins, typically in the form of pharmaceutical compositions, e.g., vaccine compositions, comprising a physiologically acceptable carrier and/or an immunostimulant. The fusion proteins may comprise multiple immunogenic polypeptides or portions/variants thereof, as described herein, and may further comprise one or more polypeptide segments for facilitating the expression, purification and/or immunogenicity of the polypeptide(s).

Within other illustrative embodiments, a polypeptide may be a fusion polypeptide that comprises multiple polypeptides, such as the lipophilin-like polypeptides as described herein, or that comprises at least one lipophilin-like polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags that facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements (an expression “control sequence”). The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91,1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A.

Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292,1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide, such as sthe lipophilin-like polypeptides of the present invention, to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Lipophilin-like polypeptides of the present invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Antibodies and Fragments Thereof

The present invention further provides agents, such as antibodies and antigen-binding fragments thereof, that specifically bind to a lipophilin complex. As used herein, an antibody, or antigen-binding fragment thereof, is said to “specifically bind” to a complex if it reacts at a detectable level (within, for example, an ELISA) with the complex. Certain preferred antibodies are mammaglobin-specific (i.e., bind to mammaglobin, preferably in its glycosylated form, and do not detectably bind free lipophilin A, lipophilin B or lipophilin C under similar conditions). Other antibodies that may be used within certain diagnostic methods provided herein specifically bind to lipophilin A, lipophilin B or lipophilin C. Antibody “binding” refers to a noncovalent association between two separate molecules. The ability to bind may be evaluated by, for example, determining a binding constant for the association. The binding constant is the value obtained when the concentration of the complex is divided by the product of the component concentrations, and may be determined using methods well known in the art. In general, an antibody is said to “bind” to a complex when the binding constant for non-covalent association with the complex exceeds about 10³ L/mol.

Binding agents may be further capable of differentiating between patients with and without a cancer, such as breast, ovarian or prostate cancer, using the representative assays provided herein. In other words, antibodies or other binding agents that bind to a lipophilin complex will generate a signal (within at least one of the methods provided herein) indicating the presence of a cancer in at least about 20% of patients with the disease, and will generate a negative signal indicating the absence of the disease in at least about 90% of individuals without the cancer. To determine whether a binding agent satisfies this requirement, biological samples (e.g., blood, sera, urine, sputum and/or tumor biopsies) from patients with and without a cancer (as determined using standard clinical tests) may be assayed as described herein for the presence of complexes that bind to the binding agent. It will be apparent that a statistically significant number of samples from patients with and without the disease should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, and RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the complex is initially injected into any of a wide variety of mammals (e.g., mice, rats, rabbits, sheep or goats). In this step, the complexes of this invention may serve as the immunogen without modification. Alternatively, particularly for complexes comprising relatively short polypeptides, a superior immune response may be elicited if the complex is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the complex may then be purified from such antisera by, for example, affinity chromatography using the complex coupled to a suitable solid support.

Monoclonal antibodies specific for a complex of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the complex of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the complex. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The complexes of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group that is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blaftler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

T Cells

Immunotherapeutic compositions may also, or alternatively, comprise T cells specific for lipophilin complex. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. Nos. 5,240,856; 5,215,926; WO 89/06280; WO 91/16116 and WO 92/07243). Alternatively, T cells may be derived from related or unrelated humans, non-human mammals, cell lines or cultures.

T cells may be stimulated with a lipophilin complex, or polynucleotides encoding a lipophilin complex and/or an antigen presenting cell (APC) that expresses such a complex. Such stimulation is performed under conditions and for a time sufficient to permit the generation of T cells that are specific for the complex. Preferably, the complex is present within a delivery vehicle, such as a microsphere, to facilitate the generation of specific T cells.

T cells are considered to be specific for a lipophilin complex if the T cells specifically proliferate, secrete cytokines or kill target cells coated with the complex or expressing a gene encoding some or all of the complex. T cell specificity may be evaluated using any of a variety of standard techniques. For example, within a chromium release assay or proliferation assay, a stimulation index of more than two fold, increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a lipophilin complex (˜100 ng/ml-100 μg/ml, preferably 200 ng/ml-25 μg/ml) for about 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a lipophilin complex, polynucleotide or complex-expressing APC may be CD4⁺, CD8⁺ or may comprise other T cell types. Lipophilin complex-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a lipophilin complex, polynucleotide or APC can be expanded in number either in vitro or in vivo. Proliferation of such T cells in vitro may be accomplished in a variety of ways. For example, the T cells can be re-exposed to a lipophilin complex, or a portion thereof corresponding to an immunogenic portion of such a complex, with or without the addition of T cell growth factors, such as interleukin-2, and/or stimulator cells that synthesize a lipophilin complex. Alternatively, one or more T cells that proliferate in the presence of a lipophilin complex can be expanded in number by cloning. Methods for cloning cells are well known in the art, and include limiting dilution.

T Cell Receptor Compositions

The T cell receptor (TCR) consists of 2 different, highly variable polypeptide chains, termed the T-cell receptor α and β chains, that are linked by a disulfide bond (Janeway, Travers, Walport. Immunobiology. Fourth Ed., 148-159. Elsevier Science Ltd/Garland Publishing. 1999). The α/β heterodimer complexes with the invariant CD3 chains at the cell membrane. This complex recognizes specific antigenic peptides bound to MHC molecules. The enormous diversity of TCR specificities is generated much like immunoglobulin diversity, through somatic gene rearrangement. The β chain genes contain over 50 variable (V), 2 diversity (D), over 10 joining (J) segments, and 2 constant region segments (C). The α chain genes contain over 70 V segments, and over 60 J segments but no D segments, as well as one C segment. During T cell development in the thymus, the D to J gene rearrangement of the β chain occurs, followed by the V gene segment rearrangement to the DJ. This functional VDJβ exon is transcribed and spliced to join to a Cβ. For the α chain, a Vα gene segment rearranges to a Jα gene segment to create the functional exon that is then transcribed and spliced to the Cα. Diversity is further increased during the recombination process by the random addition of P and N-nucleotides between the V, D, and J segments of the β chain and between the V and J segments in the □ chain (Janeway, Travers, Walport. Immunobiology. Fourth Ed., 98 and 150. Elsevier Science Ltd/Garland Publishing. 1999).

The present invention, in another aspect, provides TCRs specific for a lipophilin complex as disclosed herein, or for a variant or derivative thereof. In accordance with the present invention, polynucleotide and amino acid sequences are provided for the V-J or V-D-J junctional regions or parts thereof for the alpha and beta chains of the T-cell receptor which recognize tumor polypeptides described herein. In general, this aspect of the invention relates to T-cell receptors which recognize or bind polypeptides derived from lipophilin complexes and presented in the context of MHC. For example, cDNA encoding a TCR specific for a lipophilin complex peptide can be isolated from T cells specific for a lipophilin complex polypeptide using standard molecular biological and recombinant DNA techniques.

This invention further includes the T-cell receptors or analogs thereof having substantially the same function or activity as the T-cell receptors of this invention which recognize or bind lipophilin complex polypeptides. Such receptors include, but are not limited to, a fragment of the receptor, or a substitution, addition or deletion mutant of a T-cell receptor provided herein. This invention also encompasses polypeptides or peptides that are substantially homologous to the T-cell receptors provided herein or that retain substantially the same activity. The term “analog” includes any protein or polypeptide having an amino acid residue sequence substantially identical to the T-cell receptors provided herein in which one or more residues, preferably no more than 5 residues, more preferably no more than 25 residues have been conservatively substituted with a functionally similar residue and which displays the functional aspects of the T-cell receptor as described herein.

The present invention further provides for suitable mammalian host cells, for example, non-specific T cells, that are transfected with a polynucleotide encoding TCRs specific for a polypeptide described herein, thereby rendering the host cell specific for the polypeptide. The α and β chains of the TCR may be contained on separate expression vectors or alternatively, on a single expression vector that also contains an internal ribosome entry site (IRES) for cap-independent translation of the gene downstream of the IRES. Said host cells expressing TCRs specific for the polypeptide may be used, for example, for adoptive immunotherapy of breast cancer as discussed further below.

In further aspects of the present invention, cloned TCRs specific for a polypeptide recited herein may be used in a kit for the diagnosis of breast cancer. For example, the nucleic acid sequence or portions thereof, of tumor-specific TCRs can be used as probes or primers for the detection of expression of the rearranged genes encoding the specific TCR in a biological sample. Therefore, the present invention further provides for an assay for detecting messenger RNA or DNA encoding the TCR specific for a polypeptide.

Pharmaceutical Compositions

In additional embodiments, the present invention concerns formulations of one or more of the compositions disclosed herein in pharmaceutically-acceptable solutions for administration to a cell or an animal, either alone, or in combination with one or more other modalities of therapy.

It will also be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation, as described below for the purposes of illustration.

Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., 1997; Hwang et al., 1998; U.S. Pat. Nos. 5,641,515; 5,580,579 and 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Injectable Delivery

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. Nos. 5,543,158; 5,641,515 and 5,399,363 (each specifically incorporated herein by reference in its entirety). In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations will of course preferably meet sterility, pyrogenicity, and the general safety and purity standards as required by FDA Office of Biologics standards.

In other embodiment of the invention, the compositions disclosed herein are formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human.

Nasal Delivery

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. Nos. 5,756,353 and 5,804,212 (each specifically incorporated herein by reference in its entirety). Likewise, the delivery of drugs using intranasal microparticle resins (Takenaga et al, 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like.

The formation and use of liposomes is generally known to those of skill in the art (see for example, Couvreur et al., 1977; Couvreur, 1988; Lasic, 1998; which describes the use of liposomes and nanocapsules in targeted therapy for intracellular bacterial infections and diseases). Liposomes have been developed with improved serum stability and circulation half-times (see, for example, Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. Nos. 5,567,434; 5,552,157; 5,565,213; 5,738,868 and 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses or unacceptable toxicity after systemic delivery (Mori and Fukatsu, 1992).

In certain embodiments, liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. Moreover, the drug-bearing liposomes may be employed for site-specific delivery of active agents by selectively modifying the liposomal formulation.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, in other embodiments, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo for example, biodegradable polyalkyl-cyanoacrylate nanoparticles that meet these requirements are contemplated for use in the present invention. Such particles are easily made, as described, for example, by Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety.

Vaccine Compositions

In certain preferred embodiments of the present invention, the pharmaceutical compositions of the invention comprise immunogenic compositions, particularly vaccine compositions. Generally, such compositions will comprise one or more polynucleotide and/or polypeptide lipophilin complex compositions of the present invention in combination with an immunostimulant. An immunostimulant may be any substance that enhances or potentiates an immune response (antibody and/or cell-mediated) to an exogenous antigen.

Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

Illustrative vaccines may contain DNA encoding one or more of the lipophilin polypeptides as described above, such that some or all of the complex is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103,1989; Flexner et al., Vaccine 8:17-21,1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502,1993; Guzman et al., Circulation 88:2838-2848, 1993; and Guzman et al., Cir. Res. 73:1202-1207,1993. Techniques for incorporating DNA into such expression systems are well known to those of ordinary skill in the art.

The DNA encoding some or all of the lipophilin complex may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art may be employed in the vaccine compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed.

Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. Modified hepatitis B core protein carrier systems are also suitable, such as those described in WO/99 40934, and references cited therein, all incorporated herein by reference. One may also employ a carrier comprising the particulate-protein complexes described in U.S. Pat. No. 5,928,647, which are capable of inducing a class I-restricted cytotoxic T lymphocyte responses in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montamide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties. Other preferred adjuvants comprise polyoxyethylene ethers, such as those described in WO 99/52549A1.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438,1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Antigen Presenting Cells (APCs)

Any of a variety of delivery vehicles may be employed within pharmaceutical compositions and vaccines to facilitate production of an antigen-specific immune response that targets tumor cells. For example, in some embodiments, delivery vehicles will comprise antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251,1998) and have been shown to be effective as a physiological adjuvant for eliciting prophylactic or therapeutic antitumor immunity (see Timmerman and Levy, Ann. Rev. Med. 50:507-529,1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a lipophilin complex (or portion or other variant thereof) such that the complex, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the lipophilin complex, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

Cancer Therapy

In further aspects of the present invention, the compositions described herein may be used for immunotherapy of cancer, such as breast, ovarian or prostate cancer. Within such methods, pharmaceutical compositions and vaccines are typically administered to a patient in order to elicit an immune response directed against the lipophilin complex, or portion thereof, contained within the pharmaceutical compositions. As used herein, a “patient” refers to any warm-blooded animal, preferably a human. A patient may or may not be afflicted with cancer. Accordingly, the above pharmaceutical compositions and vaccines may be used to prevent the development of a cancer or to treat a patient afflicted with a cancer. A cancer may be diagnosed using criteria generally accepted in the art, including the presence of a malignant tumor. Pharmaceutical compositions and vaccines may be administered either prior to or following surgical removal of primary tumors and/or treatment such as administration of radiotherapy or conventional chemotherapeutic drugs.

Within certain embodiments, immunotherapy may be active immunotherapy, in which treatment relies on the in vivo stimulation of the endogenous host immune system to react against tumors with the administration of immune response-modifying agents (such as tumor vaccines, bacterial adjuvants and/or cytokines).

Within other embodiments, immunotherapy may be passive immunotherapy, in which treatment involves the delivery of agents with established tumor-immune reactivity (such as antibodies) that can directly or indirectly mediate antitumor effects and does not necessarily depend on an intact host immune system. For example, the complexes provided herein may be used to generate antibodies or anti-idiotypic antibodies (as described above and in U.S. Pat. No. 4,918,164) for passive immunotherapy. Such antibodies may be of particular benefit in the treatment of tumors with steroid-dependence, replacing the current steroid-related treatments with a less harsh, more directed form of treatment.

Routes and frequency of administration, as well as dosage, may vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more complexes, the amount of each complex present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a lipophilin complex may correlate with an improved clinical outcome. Such immune responses may be evaluated using assays, which may be performed using samples obtained from a patient before and after treatment.

It will be apparent that such therapies may be used alone, or in combination with other anti-cancer therapies. In particular, for certain tumors, pretreatment with steroids may increase the effectiveness of anti-complex treatment as described herein. In addition, the effect of existing cancer drugs may be enhanced by concurrent treatment with anti-complex immunotherapy. While not wishing to be bound by any particular theory, lipophilin complexes may associate with steroids and may participate in the regulation of growth of steroid-dependent cancers. Therapies directed at hormone-dependent growth regulation, such as Tamoxifen, may be affected by complex formation. The therapeutic methods provided herein may decrease the amount of complex present, thereby facilitating treatment using such existing therapies.

Methods for Detecting Cancer

The use of circulating serum markers to detect tumors and circulating micrometastases is becoming increasingly important for the early detection and diagnosis of cancer with the advent of routine ELISA, PCR and other sensitive procedures. For example, these methods have been used in diseases for which specific markers, such as prostate specific antigen for prostate cancer, are known. Mammaglobin has been shown to be overexpressed in breast cancer tumors (Watson, M. A. and Fleming, T. P., Cancer Res. 56:860-865, (1996)) and is a highly specific marker for breast cancer (Min et al., Cancer Res. 58:4581-4584, (1998)). For example, 81% of breast cancer tumors were immunopositive for mammaglobin protein expression, and 23% of tumors showed a ten-fold increase in mRNA levels above those found in normal breast cells (Watson (1996); Watson et al., Cancer Res 59:3028-3031, (1999)). Using RT-PCR techniques on peripheral blood, 0 out of 27 healthy individuals were found to be positive for mammaglobin message, while up to 49% of patients with known metastatic disease had detectable mammaglobin mRNA levels (Zach et al., J. Clin. Oncol. 17:2015, (1999)).

Mammaglobin protein is expressed as a small polypeptide of 93 amino acids in length with a predicted molecular weight of 10.5 kDa and a putative cleavage site at amino acid 19 (Watson (1996)). Mammaglobin shares some homology with lipophilin C (also known as mammaglobin B), having 52% identity on the amino acid level (Lehreret al., FEBS Lett. 432:163-167, (1998)) and has some similarity with rat prostatic binding protein component C3 and rabbit uteroglobin (Zhao et al., Biochem. Biophys. Res. Commun. 256:147-155, (1999); Becker et al., Genomics 54:70-78, (1998)). These proteins are known to exist as homo- or heterodimers which are covalently linked by disulfides (Morize et al., J. Mol. Biol. 194:725-739, (1987); Claessens et al., Mol. Cell. Endocrinol. 74:203-212, (1990)) and bind to aromatic compounds such as PCP and progesterone (Hard et al., Nat Struct. Biol. 2:983-989, (1995); Bochskanl et al., Hum. Reprod. 3:844-850, (1988)).

According to one aspect of the present invention, the lipophilin complexes described herein have utility in detection and diagnostic methods for patients having or suspected of having cancer, particularly breast, ovarian or prostate cancer. In general, a cancer may be detected in a patient based on the presence of one or more lipophilin complexes described herein, or antibodies directed thereto, in a biological sample obtained from the patient. In other words, such complexes may be used as markers to indicate the presence or absence of a cancer such as breast, ovarian or prostate cancer. In general, the presence of such a complex, or antibody thereto, at a level that is higher, for example at least three fold higher, in tumor tissue than in normal tissue is indicative of a cancer. The presence of such a complex may further provide information useful in the selection of therapeutic options. For example, the presence of lipophilin complexes may be indicative of tumor cell refractoriness to steroid mimic treatment. In addition, the type of cancer, as well as stage information, may be derived from the type and composition of complexes found in a tumor.

There are a variety of assay formats known to those of ordinary skill in the art for using a binding agent to detect markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of complex that binds to the binding agent; and (c) comparing the level of complex with a predetermined cut-off value. Within certain embodiments, the level of complex and the levels of free complex components are assayed. A ratio of complex to free components (i.e., binding constant) may then be determined. This ratio may be indicative of the presence, or the severity, of a cancer.

In a preferred embodiment, the assay involves the use of binding agent immobilized on a solid support to bind to and remove the complex from the remainder of the sample. The bound complex may then be detected using a detection reagent that contains a reporter group and specifically binds to the bound complex. Such detection reagents may comprise, for example, an antibody that specifically binds to the complex or an antibody or other agent that specifically binds to the binding agent, such as an anti-immunoglobulin, protein G, protein A or a lectin. Alternatively, a competitive assay may be used, in which a complex is labeled with a reporter group and allowed to bind to the immobilized binding agent after incubation of the binding agent with the sample. The extent to which components of the sample inhibit the binding of the labeled complex to the binding agent is indicative of the reactivity of the sample with the immobilized binding agent.

The solid support may be any material known to those of ordinary skill in the art to which the binding agent may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay (also referred to as a capture ELISA assay). This assay may be performed by first contacting an antibody that has been immobilized on a solid support, commonly the well of a microtiter plate, with the sample, such that complex within the sample are allowed to bind to the immobilized antibody. Unbound sample is then removed from the immobilized complex/antibody and a detection reagent (preferably a second antibody capable of binding to a different site on the complex) containing a reporter group is added. The amount of detection reagent that remains bound to the solid support is then determined using a method appropriate for the specific reporter group. Within preferred embodiments, one antibody specifically binds glycosylated mammaglobin, and the other specifically binds a lipophilin protein, such as lipophilin B. In such assays, complex may be captured with one antibody (e.g., anti-lipophilin B) and detected using the other antibody (e.g., anti-mammaglobin).

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and complex is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of complex within a sample obtained from an individual with breast, ovarian or prostate cancer. Preferably, the contact time is sufficient to achieve a level of binding that is at least about 95% of that achieved at equilibrium between bound and unbound complex. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support.

The detection reagent is then incubated with the immobilized antibody/complex for an amount of time sufficient to detect the bound complex. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as breast, ovarian or prostate cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer. Alternatively, as noted above, a similar process may be used to establish a cut-off value for the ratio of complex to free components.

For certain embodiments (e.g., sandwich assays), quantitative measurements of antigen may be obtained. Within such embodiments, a standard curve may be generated. Signals obtained for antigen levels in particular samples may then be compared to the standard curve, to allow quantitation. The cut-off value within such assays may be an amount of complex indicative of the presence of breast, ovarian or prostate cancer.

In a related embodiment, the assay is performed in a flow-through or strip test format, wherein the binding agent is immobilized on a membrane, such as nitrocellulose. In the flow-through test, complexes within the sample bind to the immobilized binding agent as the sample passes through the membrane. A second, labeled binding agent then binds to the binding agent/complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of complex that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the complexes and binding agents of the present invention. The above descriptions are intended to be exemplary only. For example, it will be apparent to those of ordinary skill in the art that the above protocols may be readily modified to use complexes as described herein to detect antibodies that bind to such complexes in a biological sample. The detection of such lipophilin complex-specific antibodies may correlate with the presence of a cancer. Other preferred assay protocols include laser scanning cytometry (a microscopic technique in which cells are stained with labeled antibody) and immunohistochemical detection. Such techniques may generally be performed according to techniques known in the art. Antibodies as provided herein may further be used to facilitate cell identification and sorting in vitro, permitting the selection of cells expressing a lipophilin complex (or varying levels of lipophilin complex). Preferably, antibodies for use in such methods are linked to a detectable marker. Suitable markers are well known in the art and include radionuclides, luminescent groups, fluorescent groups, enzymes, dyes, constant immunoglobulin domains and biotin. Within one preferred embodiment, an antibody linked to a fluorescent marker, such as fluorescein, is contacted with the cells, which are then analyzed by fluorescence activated cell sorting (FACS).

In another embodiment, the above complexes may be used as markers for the progression of cancer. In this embodiment, assays as described above for the diagnosis of a cancer may be performed over time, and the change in the level of reactive complex(es) evaluated. For example, the assays may be performed every 24-72 hours for a period of 6 months to 1 year, and thereafter performed as needed. In general, a cancer is progressing in those patients in whom the level of complex detected by the binding agent increases over time. In contrast, the cancer is not progressing when the level of reactive complex either remains constant or decreases with time. Alternatively, as noted above, the ratio of complex to the product of the free components may be monitored over time to evaluate cancer progression.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications.

To improve sensitivity, assays as described herein may be combined with assays to detect other tumor-associated antigens. It will be apparent that binding agents specific for different proteins may be combined within a single assay. The selection of tumor protein markers may be based on routine experiments to determine combinations that results in optimal sensitivity. Alternatively, pretreatment with steroids may increase the sensitivity of a complex-based diagnostic method.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a lipophilin complex. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Preferred kits are those designed for use within sandwich assays. Such kits comprise two or more components for use within such assays. For example, such a kit may comprise standards for use in preparing a standard curve. Such a kit may comprise one or both antibodies for use within the assay (i.e., the capture antibody and/or signal antibody), with or without additional reagents for use in detecting complex binding. Preferably, such a kit comprises an anti-mammaglobin antibody (or fragment thereof) and an anti-Lipophilin B antibody (or fragment thereof).

The invention also relates to therapeutics for targeting the immunosuppressive and anti-inflammatory properties of mammaglobin/lipophilin complexes. Some proteins in the uteroglobin family have been reported to be immunomodulatory molecules. The unique expression pattern found for mammaglobin-like molecules and lipophilins A, B and C and related molecules and their physical properties make it likely that these molecules may have anti-inflammatory or immunosuppressive effects. These effects may be utilized by tumors to circumvent recognition by the immune system and explain the overexpression of these molecules in a number of tumors. Thus, these molecules themselves may be manufactured for use in human subjects as immunomodulators, and antibodies targeting these molecules and complexes could be used to unmask tumors and make them accessible to the immune system.

The following Examples are offered by way of illustration and not by way of limitation.

### EXAMPLES

### Example 1

### Identification of Mammaglobin/Lipophilin B Complex in Tumor Tissue

Mammaglobin was isolated from MB415 cells (American Type Culture Collection). 1.5 l of serum free culture supernatant were dialyzed against numerous changes of 10 mM Tris (pH=8) at four degrees over two days. The dialysate was loaded with an AKTA explorer 100 (Amersham) onto a 100 ml High-Q anion exchange column (Bio-Rad) and eluted with a linear gradient from 100% Buffer A (10 mM Tris pH=8.0) to 80% Buffer B (10 mM Tris pH=8.0, 1 M NaCl) followed by a step to 100% Buffer B. Fractions were run on a 4-20% SDS-PAGE gradient gel and analyzed by Western blotting. Positive fractions were pooled and dialyzed against water overnight. Desalted, pooled material was loaded with an AKTA explorer 100 on a Source 15 RPC matrix (Pharmacia) and protein was eluted using a 0 to 100% linear gradient of 10 mM ammonium acetate in water pH=7.0 to 100% acetonitrile. Again, fractions were analyzed by Western blotting and positive fractions were pooled and lyophilized. To remove residual bovine serum albumin, the lyophilized powder was redissolved in phosphate buffered saline and run over a Affi-gel Blue column (Bio-Rad). The flow-through contained most of the mammaglobin complex and was used for further studies.

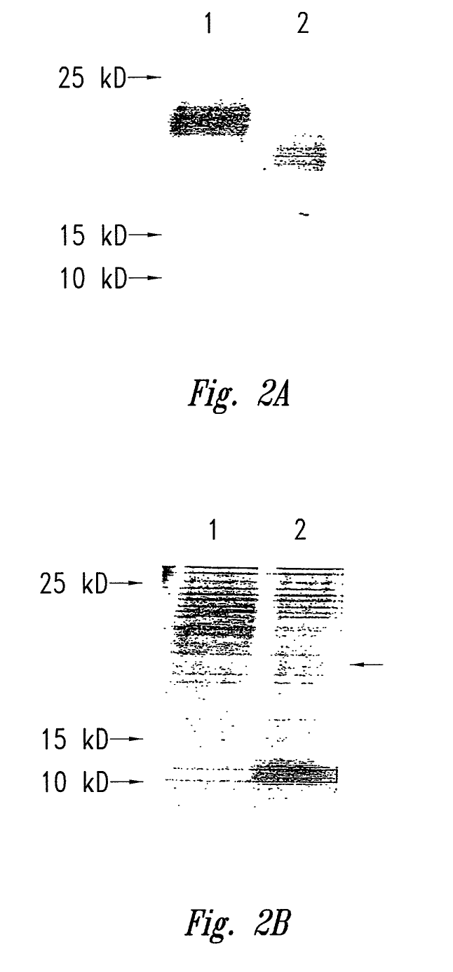

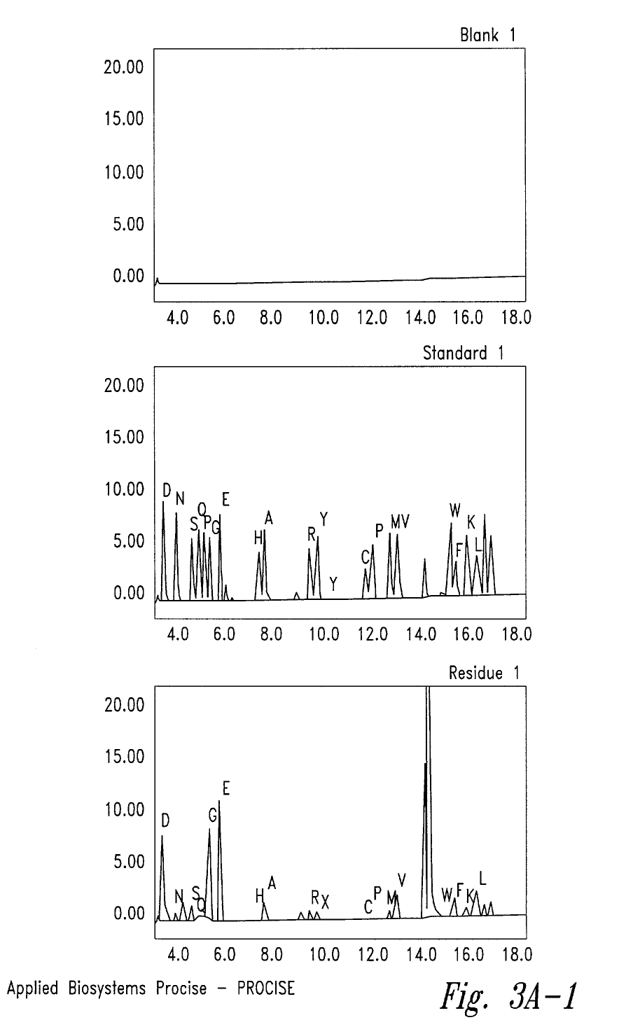

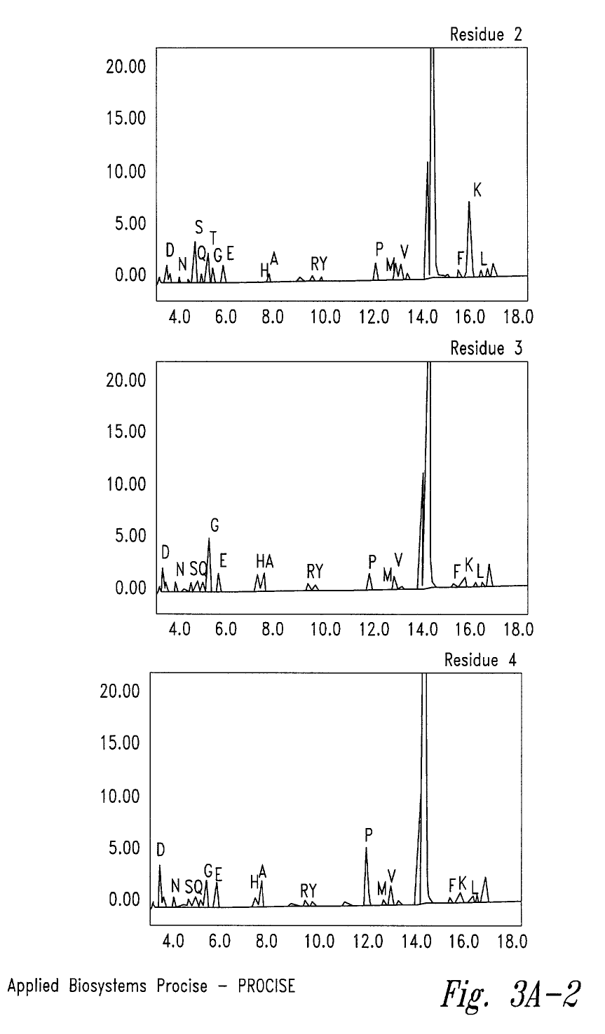

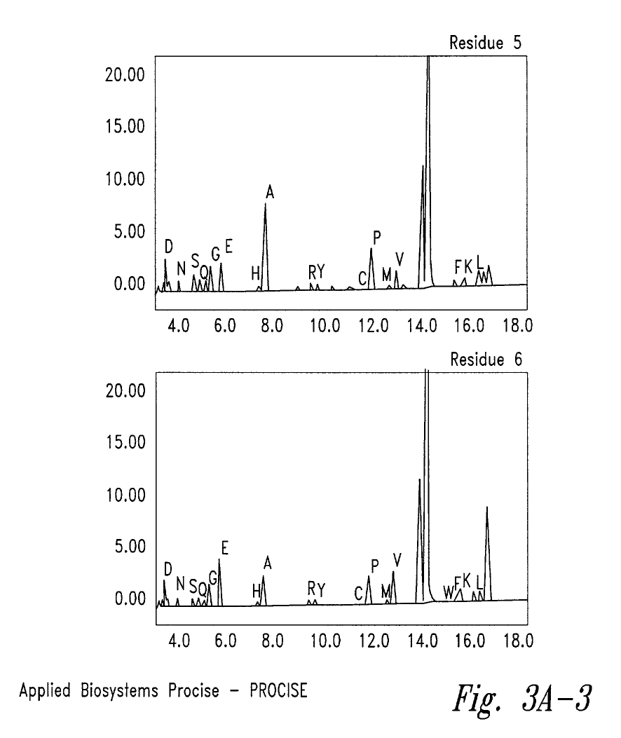

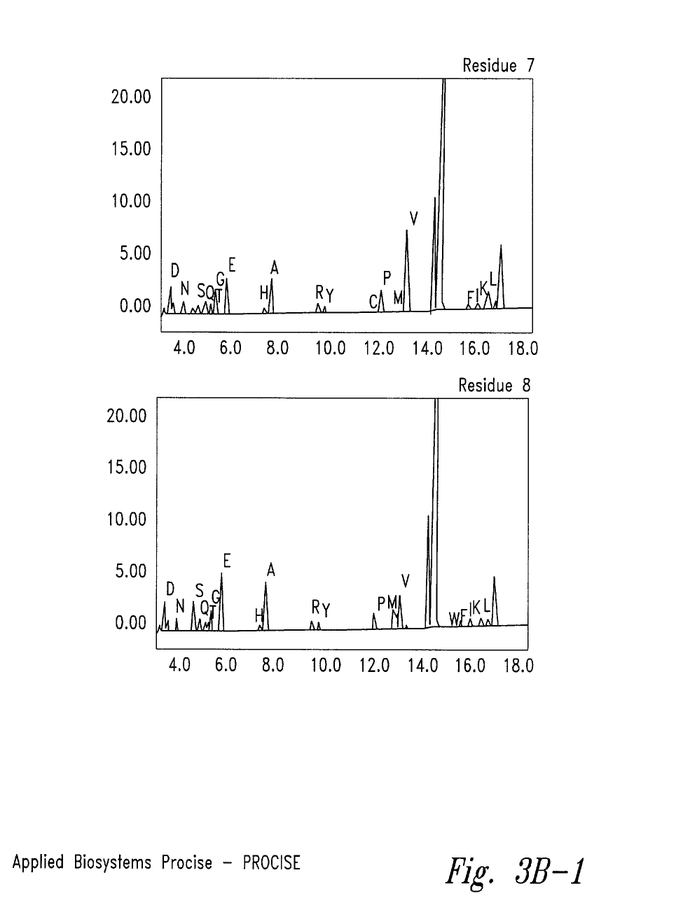

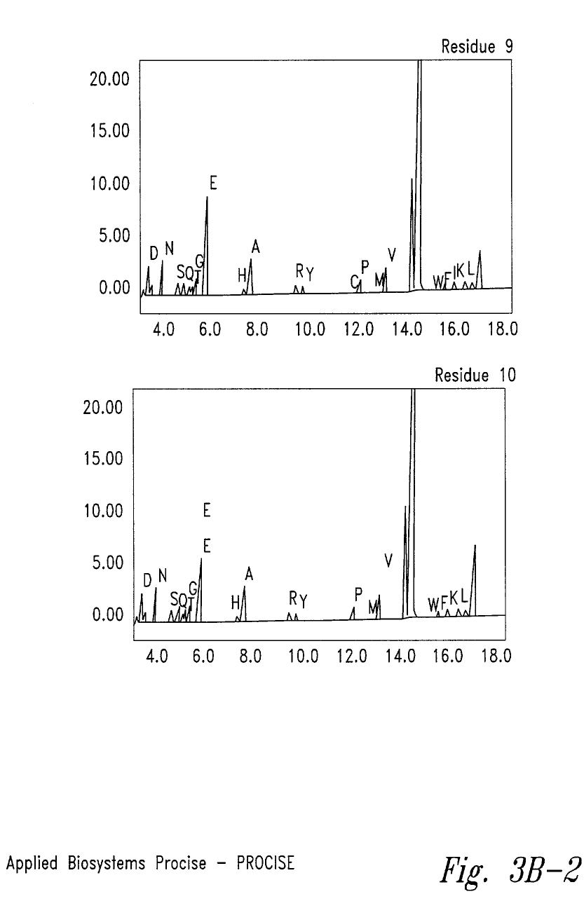

The resulting mammaglobin was analyzed on a 4-20% SDS-PAGE gel, stained with Glyco-Pro Glycoprotein Detection Kit™ (Sigma Chemical Co., St. Louis, Mo.). In FIG. 2A, lane 1 shows the complex in non-reducing SDS sample buffer, and lane 2 shows the complex with 10 mM DTT in the sample buffer. N-terminal sequencing (FIGS. 3A-3B) of an equivalent gel blotted onto PVDF membrane revealed the presence of Lipoprotein B in the band in lane 1, while no such sequence was detected in the band shown in lane 2.

FIG. 2B depicts the same gel restained with silver stain. The arrow denotes the weakly staining mammaglobin. A new band was detected in the reduced lane (lane 2), and was confirmed to be lipophilin B by N-terminal sequencing.

### Example 2

### Evaluation and Characterization of Mammaglobin/Lipophilin B Complexes in Tumor Tissue

Materials and Methods:

Chemicals and Reagents

Tris[hydroxymethyl]aminomethane (Tris), sodium chloride, ammonium acetate, phosphate buffered saline (PBS), glycine, methanol, silver nitrate, and sodium thiosulfate were obtained from Sigma (St. Louis, Mo.). Acetonitrile was from Mallinckrodt Laboratory Chemicals (Phillipsburg, N.J.).

Cell Culture

MDA-MB-415 cells (ATCC #HTB-128) cells were initially grown in DMEM (Life Technologies Inc., Rockville, Md.) supplemented with 10% fetal bovine serum (Hyclone, Logan, Utah). Once the cells were confluent, the serum containing media was removed; the cells were rinsed with PBS and low serum media was added. The low serum media consisted of a mix of 50% SFX-CHO (Hyclone, Logan, Utah) supplemented with 2 mM GlutaMAX™ (Life Technologies Inc., Rockville, Md.) and 50% Opti-MEM® 1(Life Technologies Inc., Rockville, Md.). Insulin-Transferrin-Selenium-A (Life Technologies Inc., Rockville, Md.) was added for a final concentration of 1x. The cells were grown in this media for a period of several weeks, with weekly applications of fresh media. Supernatants were collected and pooled for subsequent mammaglobin isolation.

Protein Purification

1.5 l of serum free culture supernatant were dialyzed against numerous changes of 10 mM Tris pH=8 at four degrees over two days. The dialysate was loaded with an AKTA explorer 100 (Amersham Pharmacia Biotech AB, Uppsala, Sweden) onto a 100 ml Macro-Prep High Q (Bio-Rad, Hercules, Calif.) anion exchange column and eluted with a linear gradient from 100% Buffer A (10 mM Tris pH=8.0) to 80% Buffer B (10 mM Tris pH=8.0, 1 M NaCl) followed by a step to 100% Buffer B. Fractions were run on a 4-20% SDS-PAGE gradient gel and analyzed by Western blotting. Positive fractions were pooled and dialyzed against water overnight. Desalted, pooled material was loaded with an AKTA explorer 100 onto a reversed phase column containing 15 ml of Source 15 RPC matrix (Amersham Pharmacia Biotech AB, Uppsala, Sweden) and protein was eluted using a 0 to 100% linear gradient of 10 mM ammonium acetate in water pH=7.0 to 100% acetonitrile. Again, fractions were analyzed by Western blotting and positive fractions were pooled and lyophilized. To remove residual bovine serum albumin, the lyophilized powder was redisolved in phosphate buffered saline and run over a column containing Affi-Gel Blue (Bio-Rad, Hercules, Calif.). The unbound fractions contained most of the mammaglobin protein and was used for further studies.

SDS-PAGE, PVDF- and Western Blotting

SDS-PAGE was performed according to the method of Laemmli (Laemmli, U.K. (1970) Nature 227, 680-685). Samples were diluted 1:1 in Laemmli Sample Buffer (Invitrogen, Carlsbad, Calif.) and boiled for 5 minutes. Proteins were then loaded on 4-20% acrylamide gradient gels (Bio-Rad, Hercules, Calif.) and run at 250V constant for 30 minutes. Gels were blotted by transferring to Sequi-Blot PVDF membrane (Bio-Rad, Hercules, Calif.) in 50 mM Tris base, 40 mM glycine, and 20% methanol using a Bio-Rad Trans-Blot cell. Blots were transferred for 100 minutes at 100V (constant). For Western blots, PVDF membranes were then blocked for 1 hour in phosphate buffered saline containing 0.5 M NaCl and 1% TWEEN-20 (Sigma). Next, the membranes were washed three times for ten minutes with the same buffer containing 0.1% TWEEN-20 (PBSST). Membranes were probed with 1 ug/ml of a monoclonal antibody to mammaglobin overnight and washed again as above. An horseradish peroxidase-conjugated goat anti rabbit IgG was used as a secondary antibody, developed with ECL solution (Amersham Pharmacia Biotech AB, Uppsala, Sweden), and visualized using scientific imaging film (Kodak, Rochester, N.Y.).

Protein Detection

Gels or Blots were stained either with silver (Blum et al., “Improved Silver Staining of Plant Proteins, RNA and DNA in polyacrylamide gels,” Electrophoresis 8:93-99,1987), Coomassie Brilliant Blue R250, or the Glyco-Pro glycoprotein detection kit (Sigma, St Louis, Mo.) as described (Thornton et al., “Identification of glycoproteins on nitrocellulose membranes and gels,” Walker J. M. (32), 119-128, 1994, Totowa, N.J., Humana Press, Methods in Molecular Biology).

N-Terminal Sequencing

Amino terminal sequence data were obtained from purified proteins which were dried directly onto TFA treated glass fiber filters (Perkin Elmer/Applied Biosystems Division) or from samples which were separated on SDS-PAGE and electroblotted onto Sequi-Blot PVDF membranes according to the method of Matsudaira (Matsudaira, P., J. Biol. Chem. 262:10035-10038, (1987)). The membranes were stained with the Glyco-Pro glycoprotein detection kit (Sigma, St Louis, Mo.) to visualize the protein bands that were excised with a clean razor. Traditional Edman degradation sequence analysis was performed using a Perkin Elmer/Applied Biosystems Division Procise 494 Protein Sequencer.

Quantitative Real-Time PCR

The specificity and sensitivity of the different genes was determined using quantitative PCR analysis. Breast metastases, primary breast tumors, benign breast disorders and normal breast tissue along with other normal tissues and tumors were tested in quantitative (“Real time”) PCR. This was performed either on the ABI 7700 Prism or on a GeneAmp® 5700 sequence detection system (PE Biosystems, Foster City, Calif.). The 7700 system uses a forward and a reverse primer in combination with a specific probe with a 5′fluorescent reporter dye at one end and a 3′ quencher dye at the other end (Taqman™). During PCR using the Taq DNA polymerase with 5′-3′ nuclease activity the probe is cleaved and begins to fluoresce allowing the reaction to be monitored by the increase in fluorescence (Real-time). The 5700 system uses SYBR® green, a fluorescent dye, that only binds to double stranded DNA, and the same forward and reverse primers as the 7700 instrument. Matching primers and fluorescent probes were designed for each of the genes according to the primer express program (PE Biosystems, Foster City, Calif.).The primers used for mammaglobin detection were:

| | |
|---|---|
| Mamm 2f: | |
| 5′-TGCCATAGATGAATTGAAGGAATG-3′; and | (SEQ ID NO:3) |
| Mamm 2r: | |
| 5′-TGTCATATATTAATTGCATAAACACCTCA-3′. | (SEQ ID NO:4) |

For lipophilin B, the primers were:

| | |
|---|---|
| LipoBf: | |
| 5′-TGCCCCTCCGGAAGCT-3′; and | (SEQ ID NO:5) |
| LipoBr: | |
| 5′-CGTTTCTGAAGGGACATCTGATC-3′. | (SEQ ID NO:6) |

Primers and probes so produced were used in the universal thermal cycling program in real-time PCR. They were titrated to determine the optimal concentrations using a checkerboard approach. A pool of cDNA from target tumors was used in this optimization process. The reaction was performed in 25 μl volumes. The final probe concentration in all cases was 160 nM. dATP, dCTP and dGTP were at 0.2 mM and dUTP at 0.4 mM. Amplitaq gold and Amperase UNG (PE Biosystems, Foster City, Calif.) were used at 0.625 units and 0.25 units per reaction. MgCl₂ was at a final concentration of 5 mM. Trace amounts of glycerol, gelatin and Tween 20 (Sigma Chem Co, St Louis, Mo.) were added to stabilize the reaction. Each reaction contained 2 μl of diluted template. The cDNA from RT reactions prepared as above is diluted 1:10 for gene of interest and 1:100 for β-Actin. Primers and probes for β-Actin (PE Biosystems, Foster city, Calif.) used in a similar manner to quantitate the presence of β-actin in the samples. In the case of the SYBR® green assay the reaction mix (25 μl) included, 2.5 μl of SYBR green buffer, 2 μl of cDNA template and 2.5 μl each of the forward and reverse primers for the gene of interest. This mix also contained 3 mM MgCl₂, 0.25 units of AmpErase UNG, 0.625 units of Amplitaq gold, 0.08% glycerol, 0.05% gelatin, 0.0001% Tween 20 and 1 mM dNTP mix. In both formats, 40 cycles of amplification were performed. In order to quantify the amount of specific cDNA (and hence initial mRNA) in the sample, a standard curve is generated for each run using a plasmid containing the gene of interest. Standard curves were generated using the Ct values determined in the real-time PCR which were related to the initial cDNA concentration used in the assay. Standard dilutions ranging from 20-2×10⁶ copies of the gene of interest were used for this purpose. In addition, a standard curve is generated for the housekeeping gene β-actin ranging from 200 fg-200 pg to enable normalization to a constant amount of β-Actin. This allows the evaluation of the over-expression levels seen with each of the genes. (Holland et al., Proc. Natl. Acad. Sci. U.S.A. 88:7276-7280, (1991); Schneeberger et al., PCR Methods Appl. 4:234-238, (1995)).

Results:

Transcription of Mammaglobin and Lipophilin B in Various Tissues

Real time PCR was used to examine the message levels of mammaglobin and lipophilin B in a number of breast tumors, other tissues, and tumor cell lines. These data demonstrated an association between mRNA expression of lipophilin B and mammaglobin in the majority of breast tumors with a Spearman Rank Correlation coefficient (Rosner, B. Fundamentals of Biostatistics. Payne, M R., Hankinson S., and London S. 3rd, 451-453. 1990. USA, PWS-KENT Publishing Co) of 79% for 24 tumor samples (p=0). The same coefficient, calculated for another breast-linked marker, Her2/neu, for which there is no evidence of linkage to mammaglobin, and lipophilin B is 16% for the 24 samples (p<=0.42).

Purification of the Complex

Mammaglobin-lipophilin B was purified to greater than 90% purity as assayed by N-terminal sequencing of the purified liquid pool after reverse phase chromatography. Due to the poor staining of the complex by conventional means this was the best measure to use to assay purity. To verify that there was no significant contaminant with a blocked N-terminus, a blot was stained by Coomassie Brilliant Blue and the only visible other band sequenced. This band was not N-terminally blocked and was revealed to be serum albumin left from the cell culture medium.

Mammaglobin Glycosylation

Purified mammaglobin stained easily by a carbohydrate specific stain. Fine tuning of the acetonitrile gradient used to purify the complex revealed a sequence of peaks cumulating on one major final peak. These peaks were identified all as having a major mammaglobin component by Western blotting (data not shown) and are interpreted as differentially glycosylated forms of mammaglobin with the predominant form being the final product of glycosylation.

Mammaglobin—Lipophilin B Association

Mammaglobin and lipophilin B copurified through numerous different biochemical purification steps. They also co-migrated on SDS-PAGE gels under non-reducing conditions. When blotted on PVDF membranes and sequenced, the diffuse band which contained mammaglobin by Western blotting also contained equimolar amounts of lipophilin B. This association of mammaglobin and lipophilin B can be broken by pre-treatment with reducing agents such as 10 mM dithiothreitol indicating an association by disulfide linkage. Prior to reduction, the center of the mammaglobin band was at about 25 kD on SDS-PAGE gels. After reduction, this band shifted downward to about 20 kD, consistent with a loss of one molecule of lipophilin B per complex (predicted molecular mass: 7.6 kD). Below the 10 kD molecular weight marker a new band appeared upon reduction which was absent in the non-reduced gels. This band could be stained using conventional silver staining and was revealed to be lipophilin B by N-terminal sequencing. A summary of the N-terminal sequences of the mammaglobin preparations is set forth in Table 1.

TABLE 1
N-Terminal Sequences of Mammaglobin Preparations

| Material Sequenced | N-terminal sequence | Identification |
|---|---|---|
| purified mammaglobin | EFxPALV . . . | Lipophilin B (45%) |
| protein | GSGxPxL . . . | Mammaglobin (45%) |
| | DTH . . . | Serum Albumin (10%) |
| 25 kD band in non-reduced | EFxPALVSEL . . . | Lipophilin B (50%) |
| gel | GSGxPxLENV . . . | Mammaglobin (50%) |
| 20 kD band in reduced gel | GSGxPLL . . . | Mammaglobin (100%) |
| <10 kD band in reduced | EFxPALV . . . | Lipophilin B (100%) |
| gel | | |
| contaminant band | DTHKSEI . . . | Serum Albumin (80%) |
| | | some other low signals |
| | | in this band |

The N-terminal sequences determined are consistent with mammaglobin and lipophilin B being processed at their respective predicted cleavage sites, between amino acids 19 and 20 for mammaglobin; and amino acids 21 and 22 for lipophilin B.

Thus, mammaglobin, a diagnostic marker for breast cancer, has been found to be associated with lipophilin B. Previously not known as a breast cancer marker, lipophilin B in its association with mammaglobin may be a good serological marker for breast cancer. The significant association found in breast tumors using a rank correlation coefficient, which assumes nothing about distribution of values or the linearity of the association, indicates a significant paralleling of message expression for both mammaglobin and lipophilin B.

In rat, homologous proteins found in the rat prostatic binding protein complex are also know to be attached to each other (Claessens et al., Mol. Cell Endocrinol. 74:203-212, (1990)). Specifically, there are two heterodimers: the C1-C3 heterodimer; and the C2-C3 heterodimer. C1 and C2 are homologous to Lipophilins A and B, while C3 is homologous to Mammaglobin and Lipophilin C (also known as Mammaglobin B). These sequence comparisons are summarized below in Table 2.

TABLE 2
Homology of different uteroglobin-like molecules

| | | Mamm. | Rat | Lipo. | Lipo. | Rat | Rat |
|---|---|---|---|---|---|---|---|
| ° | Mamm. | B | C3 | A | B | C1 | C2 |
| Mammaglobin | — | | | | | | |
| Mammaglobin B | 52% | — | | | | | |
| Rat PBP C3 | 43% | 36% | — | | | | |
| Lipophilin A | 0* | 0* | 0* | — | | | |
| Lipophilin B | 0* | 0* | 0* | 53% | — | | |
| Rat PBP C1 | 0* | 0* | 0* | 35% | 44% | — | |
| Rat PBP C2 | 0* | 0* | 0* | 38% | 40% | 39% | — |
| Rabbit | 24% | 0* | 24% | 25% | 20% | 19% | 20% |
| Uteroglobin | | | | | | | |

Protein sequences without their signal peptides were aligned using the BLASTP algorithm (20) and a PAM250 scoring matrix. Percent identity is reported between family members.
* = no significant homology was detected using a low stringency expect value of 1000.

Pairwise sequence alignment of these different proteins sorts them into two groups: mammaglobin-like molecules, and lipophilin-like molecules. For each mammaglobin-like molecule, a dimerization partner has been detected, except for mammaglobin itself (Lehrer et al., FEBS Lett. 432:163-167, (1998); Claessens et al., Mol. Cell Endocrinol. 74:203-212, (1990)). This suggests a modular formation of these complexes in which a lipophilin-like protein can complex to a mammaglobin-like protein. For example, research in human tears has found that Mammaglobin B and Lipophilin A are associated by disulfide bonds (Lehrer et al., FEBS Lett 432:163-167, (1998)). In rat prostatic binding protein, mammaglobin-like C3 is complexed to lipophilin-like C1 and C2, and in rat lacrimal gland the C3 component is disulfide bonded to another as yet unidentified 10 kD polypeptide which is distinct from either C1 or C2 (Vercaeren et al., Endocrinology 137:4713-4720, (1996)). Thus, different associations between lipophilin-like molecules and mammaglobin-like molecules likely exist that are specific for the type of tissue and the function to be exerted by these complexes.

Moreover, regulation of transcription of these molecules in humans by steroids can be postulated. In rats, C3 message is highly regulated by androgens both in the prostate and in the lacrimal gland. If this is also true in humans, then up-regulation of the messages in breast cancer tissues may be an indicator of steroid responsiveness for tumors expressing the mammaglobin-lipophilin B complex.

### Example 3

### Detection of Antibodies to Lipophilin B in Breast Cancer Sera

An ELISA procedure was carried out using the following reagents:

| 1 × PBS (1 liter) | | Wash Solution | |
|---|---|---|---|
| Na₂HPO₄ | 1.42 g | 1 × PBS | 1 liter |
| NaH₂PO₄ | 1.20 g | Tween 20 | 1 ml |
| NaCl | 8.5 g | pH to 7.4 | |
| bring dH₂O up to 1 liter | | | |
| pH to 7.4 | | | |
| Blocking Buffer | | Coating Buffer | |
| (5% non-fat milk/PBS) | | Na₂CO₃ | 1.6 g |
| Non-fat dried milk | 25 g | NaHCO₃ | 2.9 g |
| PBS | 500 mls | bring up to 1 liter with dH₂O | |
| | | pH to 9.6 | |
| Serum Diluent | | HRP-Protein A | |
| (5% non-fat milk/PBS/0.1% Tween) | | ZYMED (purchased through VWR) | |
| Non-fat dried milk | 25 g | catalog # 10-1023 | |
| PBS | 500 mls | | |
| Tween 20 | 500 ul | | |
| Conjugate Diluent | | TMB Microwell Peroxidase Substrate | |
| (0.1% Tween/0.1% BSA/PBS) | | Kirkegaard & Perry Laboratorie | |
| Tween 20 | 1 ml | catalog # 50-76-05 | |
| BSA | 1 g | | |
| PBS | 1 liter | | |
| | | 1 N H₂SO₄ | |

Procedure

The buffers were allowed to come to room temperature. Plates were coated the night before and incubated overnight at 4° C. 50 μl per well of antigen was used, diluted in coating buffer. (The plate can be coated the same day at 37° C. for 1 hour.) Recombinants were coated at 200 ng/well, Peptides at 1 ug/well. Plates were aspirated, and 250 μl of Blocking Buffer was added to each well and incubated for 2 hours. A 1:100 dilution of serum was made in serum diluent using 50 ul/well. Plates were washed at 350 μl per well and washed 6 times. 50 ul/well of serum dilutions was added to the plates and incubated 2 hours at room temperature, then washed. 50 μl per well of a 1:60,000 dilution of HRP-Protein A in conjugate diluent (dilution depends on the strength of the HRP-Protein A) was added, and incubated 1 hour at room temperature. The plates were washed as described. 100 μl of TMB Microwell Peroxidase Substrate was added to each well and incubated 15 minutes in the dark at room temperature. The reaction was stopped with 100 μl N H₂SO₄ and the plates were read immediately at 450 nm. The mean and standard deviation of the normals were calculated. To calculate the cutoff, the standard deviation was multiplied by three and the mean was added. Anything above this number was positive and anything below this number was negative.

Results

In an ELISA assay, as described above, with Lipophilin Bcoated on the plate, antibodies were detected in 9 out of 24 stage 4 breast cancer sera. Lower levels of reactivity were detected in stage 2 and 3 breast cancer sera. There is some indication that antibodies may be detected in ovarian cancer sera. No antibodies were detected in prostate, lung, endometrial cancer sera, nor in normal human sera.

### Example 4

### Mammaglobin Linkage to Lipophilin B Via Cysteine Bridge

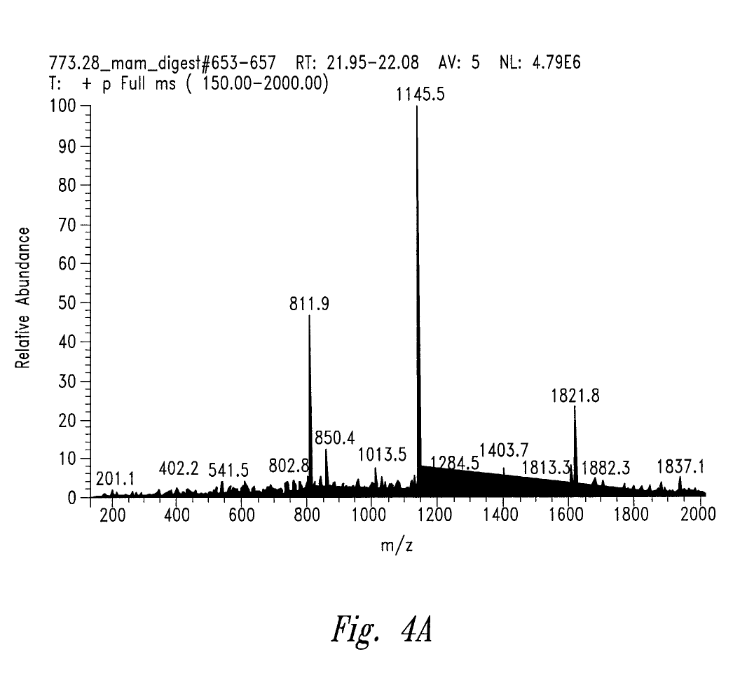

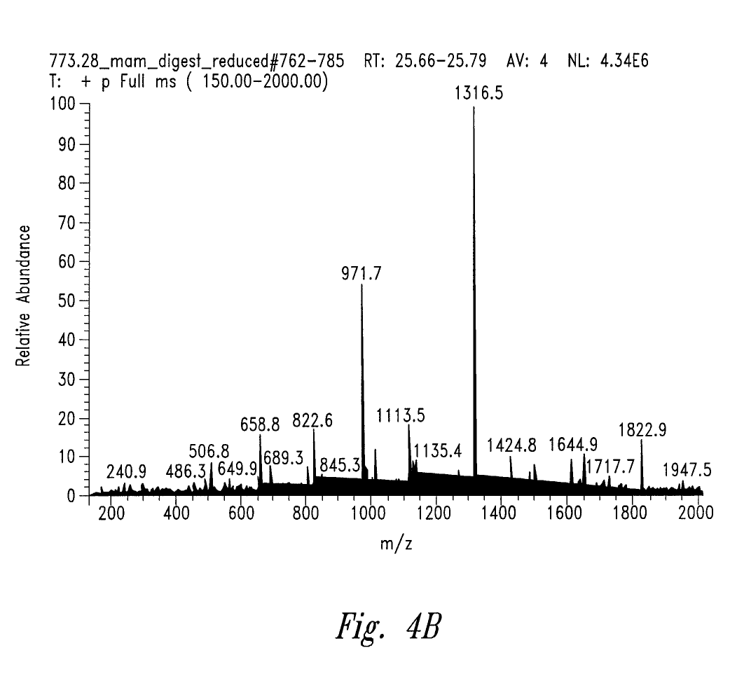

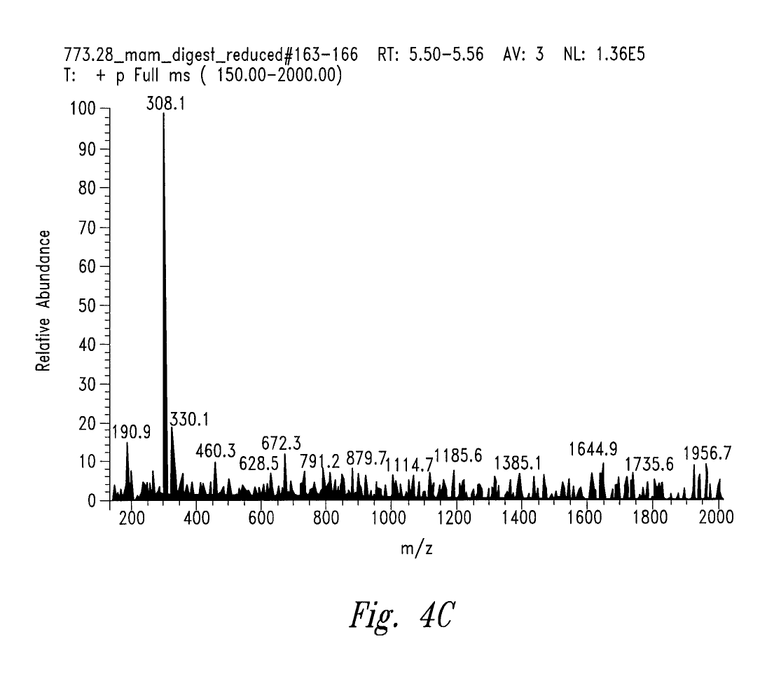

Mass spec data indicate that mammaglobin purified from MB415 cells is covalently linked to lipophilin B via a cysteine bridge from mammaglobin amino acid 4 to lipophilin amino acid 67. The results of the mass spec analysis are shown in FIG. 4. The 1621.6 ion matches the combined molecular weight of mammaglobin tryptic fragment 1-13 and lipophilin B tryptic fragment 67-69. As shown in FIG. 4, mammaglobin tryptic fragment 1-13 (MW 1316.5) and lipophilin fragment 67-69 (MW 308.1) are visible.

Mammaglobin lipophilin B complex purified from MB415 cells was subjected to digestion with trypsin. Fragments were analyzed by mass spectrometry (MS/MS). In addition to the predicted tryptic fragments, two fragments at +14 Daltons were identified. MS/MS data from th mammaglobin tryptic fragment of amino acids 1-13 indecated that amino acids 8, 9 or 10 were modified in such a manner as to cause a +14 Dalton shift in the predicted molecular weight of the tryptic fragment. Similar analysis of lipophilin B tryptic fragment comprised of amino acids 54-62 indicated that a similar polymorphism occurred between amino acids 54 and 59.

These data suggest that polymorphisms in mammaglobin and lipophilin may be used in diagnostic and prognostic applications. Specific therapeutics and drugs may be targeted to these polymorphisms and, consequently, may find utility in the development of vaccines.

### Example 5

### Variants of Mammoglobin and Lipophilin B

Breast tumors from multiple patients, and single normal breast sample (S11), and the cell line MB415 were used to derive cDNA. These were used to PCR amplify portions of the mammaglobin and lipophilin B cDNAs that included the open reading frames. These were then cloned and sequenced. These sequences demonstrate the existence of nucleic acid changes (either polymorphisms or mutations), some of which result in a change in the amino acid composition of the proteins.

As set forth in Table 3 below, the polynucleotides encoding the mammaglobin reference molecule and the above-mentioned variants are included in SEQ ID NO:18-26, and the amino acid sequences encoded by these are included in SEQ ID NO:28-34 (see also SEQ ID NO:1 for the mammaglobin reference protein). The polynucleotides encoding the lipophilin B reference molecule and variants are included in SEQ ID NO:7-17, and the amino acid sequences are included in SEQ ID NO:35-42 (see also SEQ ID NO:2 for the lipophilin B reference protein). Note that not all of the polynucleotide variants result in an amino acid change in the encoded protein.

TABLE 3

| Sequence Name | SEQ ID NO |
|---|---|
| Lipophilin B S11 3 3 | 7 |
| Lipophilin B MB415 | 8 |
| Lipophilin B 457A 5 | 9 |
| Lipophilin B SEQ | 10 |
| Lipophilin B 395A 2 | 11 |
| Lipophilin B 392A 2 | 12 |
| Lipophilin B 318A 3 | 13 |
| Lipophilin B 207A 4 | 14 |
| Lipophilin B 207A 3 | 15 |
| Lipophilin B 154A 3 | 16 |
| Lipophilin B 395A 4 | 17 |
| Mammaglobin 207A 2 61551.2 | 18 |
| Mammaglobin S443A 3 61572.2 | 19 |
| Mammaglobin S443A 2 61571.2 | 20 |
| Mammaglobin S392A 5 61569.2 | 21 |
| Mammaglobin S11 3 4 61563.2 | 22 |
| Mammaglobin S11 3 2 61561.2 | 23 |
| Mammaglobin MB415 5 61549.2 | 24 |
| Mammaglobin 446A 2 61556.2 | 25 |
| Mammaglobin 207A 5 61554.2 | 26 |
| Mammaglobin cDNA SEQ | 27 |
| Mammaglobin 207A 2 4aa | 28 |
| Mammaglobin S443A 2aa | 29 |
| Mammaglobin S11 3 4aa | 30 |
| Mammaglobin S11 3 2aa | 31 |
| Mammaglobin 446A 2 6aa | 32 |
| Mammaglobin 392A 5aa | 33 |
| Mammaglobin ORF | 34 |
| Lipophilin B ORF | 35 |
| Lipophilin B S11 3 3aa | 36 |
| Lipophilin B MB415aa | 37 |
| Lipophilin B 457A 5aa | 38 |
| Lipophilin B 207A 4aa | 39 |
| Lipophilin B 392A 2aa | 40 |
| Lipophilin B 318A 3aa | 41 |
| Lipophilin B 395A 4aa | 42 |

### Example 6

### Identification of Additional Variants of Mammoglobin

Mammaglobin cDNA was amplified from 8 primary breast tumors, 10 metastatic breast tumors, and a single normal breast tissue sample. These were then subcloned and sequenced as described in Example 5 to determine the extent of sequence variants that exist. The sequence variants identified are summarized in Table 4. The DNA sequences are disclosed in SEQ ID NOs:43-50, and 55 and the amino acid sequence of those variants resulting in amino acid changes are disclosed in SEQ ID NOs:51-54.

| | | | Description of sequence variants isolated (in |
|---|---|---|---|
| Tissue/Tumor | | Number of | comparison to SEQ ID NO: 1 for amino acid variants and |
| Source | RNA ID | Sequences | SEQ ID NO: 27 for DNA variants) |
| Breast Met | 595A | 5 | Variant 1-3 aa deletion (79-81) (DNA SEQ ID NO: 50; |
| | | | Amino acid SEQ ID NO: 54) |
| | | | Variant 2-A-> G in 3′ UTR (bp 334) (DNA SEQ ID |
| | | | NO: 46) |
| | | | 3 wild type sequences |
| Breast Met | 612A | 5 | All sequences were wild type |
| Breast Met | 613A | 4 | All sequences were wild type |
| Breast Met | 614A | 5 | All sequences were wild type |
| Breast Met | 615A | 4 | All sequences were wild type |
| Breast Met | 618A | 5 | Variant 1-A-> T in 5′ UTR (bp −23) (DNA SEQ ID |
| | | | NO: 45) |
| | | | Variant 2-A-> T at bp 217 (Thr-> Ser at aa 73) (DNA |
| | | | SEQ ID NO: 44; Amino acid SEQ ID NO: 52) |
| | | | 3-wild type sequences |
| Breast Met | 619A | 5 | Variant 1-G-> T at bp 3 (Met-> Ile at aa 1) (DNA SEQ |
| | | | ID NO: 43; Amino acid SEQ ID NO: 51) |
| | | | Variant 2-A-> G in 3′ UTR (334) (same as DNA SEQ ID |
| | | | NO: 46) |
| | | | 3-wild type sequences |
| Breast Met | 620A | 5 | All sequences were wild type |
| Breast Met | 627A | 4 | All sequences were wild type |
| Breast Met | 629A | 5 | All sequences were wild type |
| Primary Breast | 10A | 5 | All sequences were wild type |
| Tumor | | | |
| Primary Breast | 392A | 3 | All sequences were wild type |
| Tumor | | | |
| Primary Breast | 467A | 5 | Variant 1-C-> T bp 161 (Ala-> Val at aa 54) (DNA SEQ |
| Tumor | | | ID NO: 48; Amino acid SEQ ID NO: 53) |
| | | | 4 wild type sequences |
| Primary Breast | 87C | 5 | All sequences were wild type |
| Tumor | | | |
| Primary Breast | 155A | 5 | Variant 1-3 aa deletion (79-81), T-> A in 5′ UTR |
| Tumor | | | (bp −26) (DNA SEQ ID NO: 47) |
| | | | 4 wild type sequences |
| Primary Breast | 208A | 5 | All sequences were wild type |
| Tumor | | | |
| Primary Breast | 81D | 5 | All sequences were wild type |
| Tumor | | | |
| Primary Breast | 213A | 5 | Variant 1-3 aa deletion (79-81), A-> G in 3′ UTR (bp |
| Tumor | | | 334) (DNA SEQ ID NO: 55; Amino acid seqs same as |
| | | | SEQ ID NO: 54) |
| | | | Variant 2-A-> G in 3′ UTR (bp 334) (same as DNA SEQ |
| | | | ID NO: 46) |
| | | | Variant 3-G-> A in 3′ UTR (bp 347) (DNA SEQ ID |
| | | | NO: 49) |
| | | | Variant 4-wild type |
| Normal Breast | 210A | 3 | All sequences were wild type |

### Example 6

### Construction of Mammaglobin and Lipophilin Fusion Proteins

Various Mammaglobin/Lipophilin fusion proteins with and without leader sequences are constructed. The cDNA encoding Mammaglobin and Lipophilin, each including leader sequence, are fused to yield constructs that are used in vaccine compositions comprising these complexes either as DNA, or expressed in a prokaryotic or eukaryotic system to generate a recombinant protein-based vaccine. The following fusions are constructed using standard molecular biological techniques: Mammaglobin-Lipophilin including signal sequences for both molecules, Lipophilin-Mammaglobin also including both signal sequences, Mammaglobin-Lipophilin with no signal sequences, and Lipophilin-Mammaglobin with no signal sequence (for example as disclosed in the cDNAs set forth in SEQ ID NOs:56, 57, 58, and 59, respectively, and the predicted amino acid sequences set forth in SEQ ID NOs:60, 61, 62, and 63, respectively). Standard techniques may be used as described for example in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.:

### Example 7

### Construction of Mammaglobin-B305D Breast Tumor Antigen Fusion Proteins

B305D is a breast tumor antigen isolated by differential display PCR (see U.S. application Ser. No. 08/585,392 filed Jan 11,1996, and Ser. No. 09/062,451 filed Apr. 17, 1998). RT-PCR showed B305D to be over-expressed in 75% of breast tumors. It is also overexpressed in prostate tumor, normal prostate, and testis. Mammaglobin-B305D fusion constructs with and without signal sequences are constructed for use either as a DNA vaccine or expressed in a prokaryotic or eukaryotic system to generate a recombinant protein-based vaccine. For example, the polynucleotide encoding Mammaglobin without a leader sequence is fused to the B305D C form gene (as disclosed in the cDNA set forth in SEQ ID NO:64 and the predicted amino acid sequence set forth in SEQ ID NO:66) or the polynucleotide encoding the full length Mammaglobin with its leader sequence is fused to the full length B305D C form breast tumor gene (as disclosed in the cDNA sequence set forth in SEQ ID NO:65 and the predicted amino acid sequence set forth in SEQ ID NO:67). These fusion molecules are constructed using standard molecular biological techniques known in the art. Fusions are constructed that also include N-terminus and C-terminus tags (for example a 6 histidine tag) for use in purification and/or detection of recombinant protein.

The expression level of B305D is enhanced by fusing it to mammaglobin. The immunogenicity of the smaller mammaglobin protein is enhanced by fusion to the B305D C form protein. The fusion molecule allows treament of a greater percentage of patients with breast cancer, since approximately 80-90% of breast tumors express at least one of these genes.

### Example 8

### Generation of Polyclonal Antibodies to Lipophilin B

This Example describes the production of polyclonal rabbit antibodies to the lipophilin B protein.

The lipophilin B protein used in the production of polyclonal antibodies was made by peptide synthesis and frozen until needed for immunization.

Generation of Polyclonal Antisera

400 micrograms of lipophilin B was combined with 100 micrograms of muramyldipeptide (MDP). Equal volume of Incomplete Freund's Adjuvant (IFA) was added and then mixed and injected into a rabbit. Every four weeks the rabbit was boosted with 100 micrograms of antigen mixed with an equal volume of IFA. Seven days following each boost the animal was bled. The sera was generated by incubating the blood at 4° C. for 12-24 hours, followed by centrifugation.

Characterization of Polyclonal Antisera

96 well plates were coated with lipophilin B by incubating with 50 microliters (typically 1 microgram) at 4° C. for 20 hrs. 250 microliters of BSA blocking buffer was added to the wells and incubated at RT for 2 hrs. Plates were washed 6 times with PBS/0.01% Tween. Rabbit sera were diluted in PBS. Fifty microliters of diluted sera was added to each well and incubated at RT for 30 min. Plates were washed as described above before 50 microliters of goat anti-rabbit horse radish peroxidase (HRP) at a 1:10000 dilution was added and incubated at RT for 30 min. Plates were washed as described above and 100 microliters of TMB Microwell Peroxidase Substrate was added to each well. Following a 15-minute incubation in the dark at room temperature, the colorimetric reaction was stopped with 100 microliters of 1N H₂SO₄ and read immediately at 450 nm. The polyclonal antibodies showed specific immunoreactivity with lipophilin B.

### Example 9

### Development of a Capture Elisa Assay for Mammaglobin-Lipophilin B Complexes

Rabbit anti-mammaglobin antibodies (RO28, RO48 and RO62) were generated and used to set up a capture ELISA assay (also referred to as a two-antibody sandwich assay) as described below.

Selected Lymphocyte Antibody Method (SLAM, Abgenix Biopharma, Vancouver, BC) was used to generate rabbit monoclonal antibodies from rabbits that were immunized with mammaglobin that was purified from the culture media of MDA-MB-415 breast carcinoma cells. The RO28, RO48, and RO62 antibodies were used in the Capture ELISA developed as described in detail below. R028 recognizes only the mammaglobin-lipophilin B complex. This antibody does not bind to mammaglobin alone. RO48 and RO62 bind to mammaglobin alone as well as the mammaglobin-lipophilin B complex.

96-well plates were coated with 50 microliters of each of the monoclonal antibodies that were diluted in bicarbonate buffer at 4 micrograms/ml. The plates were then incubated at 4° C. for 16 hrs. 250 microliters of blocking buffer (1×PBS+1% BSA) was added to the wells and incubated at RT for 2 hrs. Plates were washed 6 times with PBS/0.01% Tween. Serial dilutions of the recombinant mammaglobin and purified supernatant from MDA-MB-415 cells (a human breast cancer cell line that expresses mammaglobin-lipophilin complex) were made in dilution buffer (PBS/0.1%Tween/0.1%BSA). These standards were then diluted by 50% in normal human sera. Human sera samples were diluted 50% in dilution buffer. Fifty microliters of diluted mammaglobin standard and human sera was added to each well and incubated at RT for 120 minutes. Plates were washed as described above, then 50 microliters of biotinylated RO28, RO48 or RO62 antibody (at 1 microgram per milliliter) was added. The plates were incubated for 60 minutes at room temperature and washed as described. Next, 50 microliters of streptavidin-HRP at a 1:10000 dilution was added and incubated at RT for 30 minutes. Plates were washed as described above and 100 microliters of TMB Microwell Peroxidase Substrate was added to each well. Following a 15-minute incubation in the dark at room temperature, the colorimetric reaction was stopped with 100 microliters of 1N H₂SO₄ and read immediately at 450 nm.

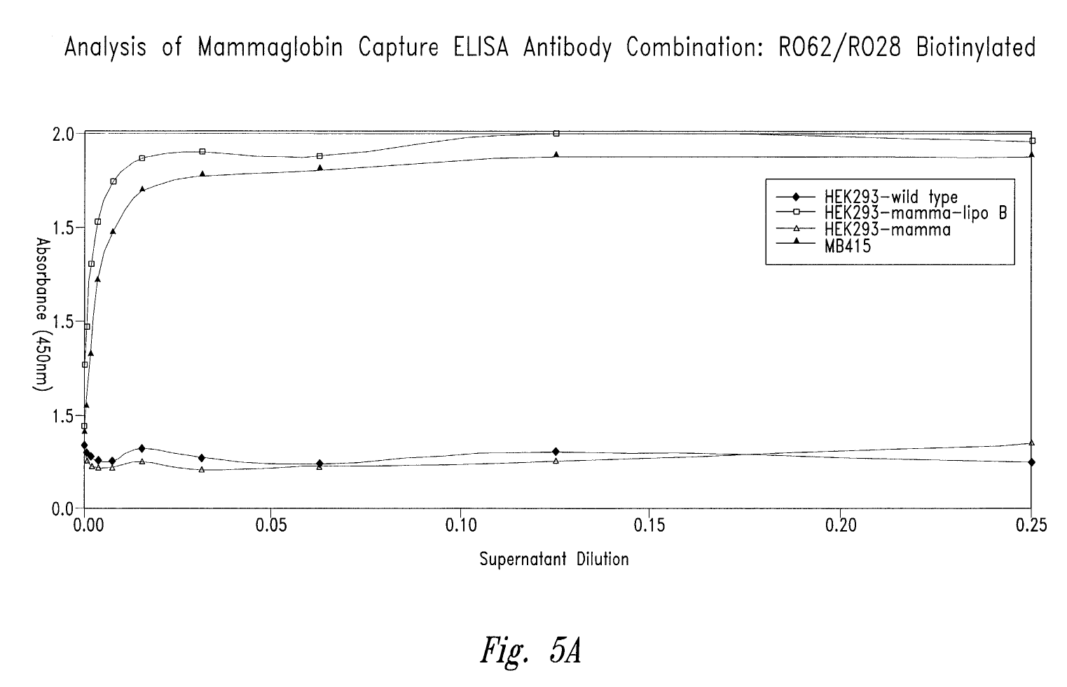

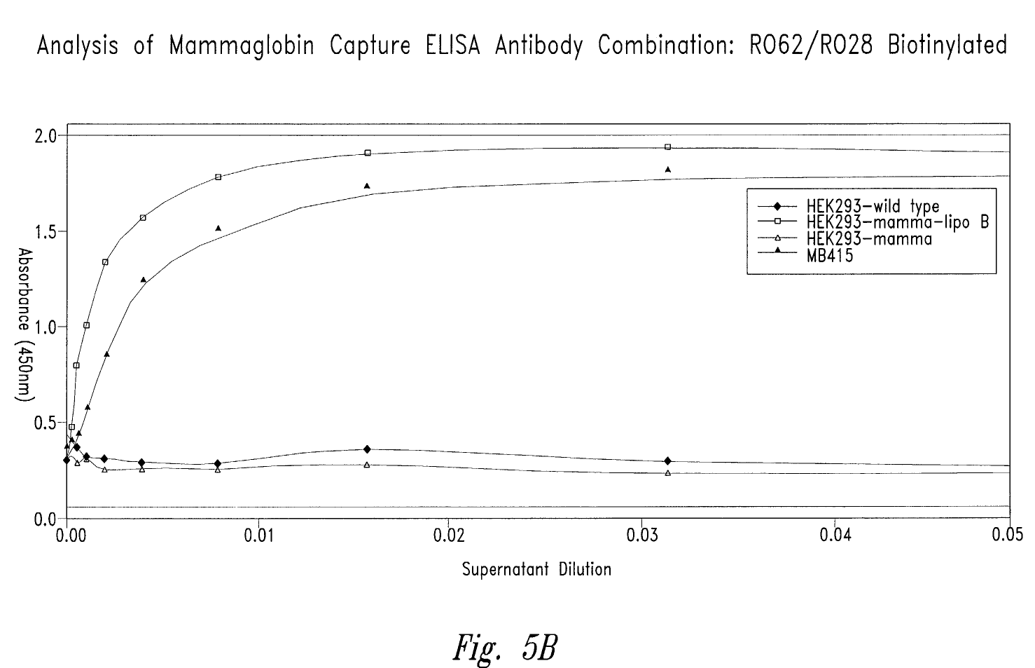

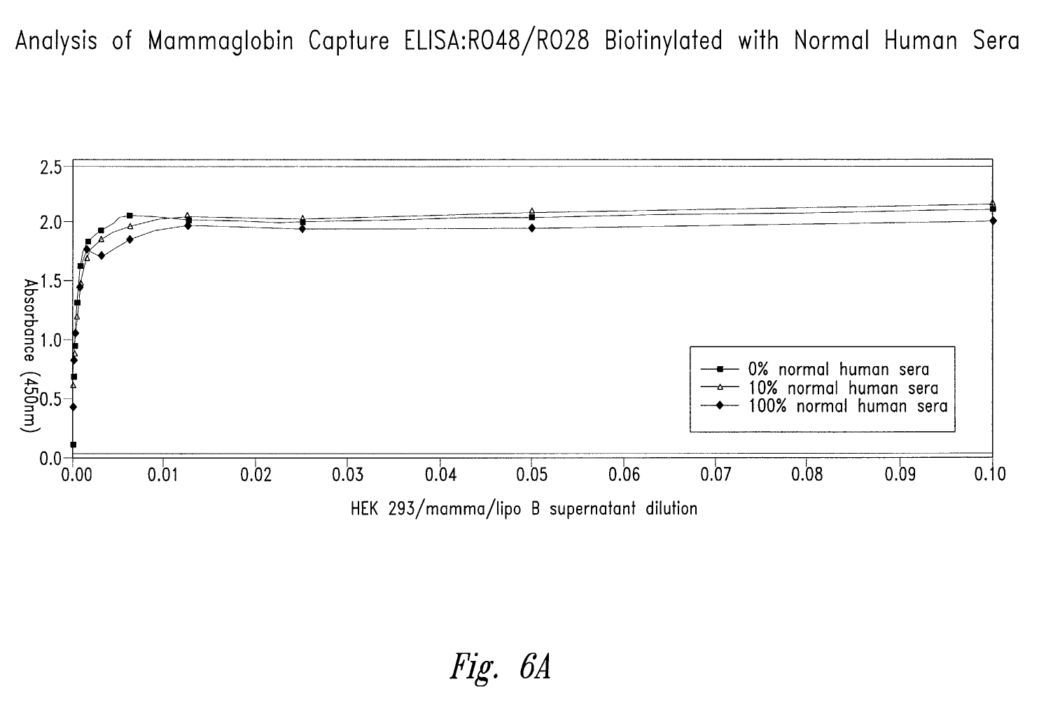

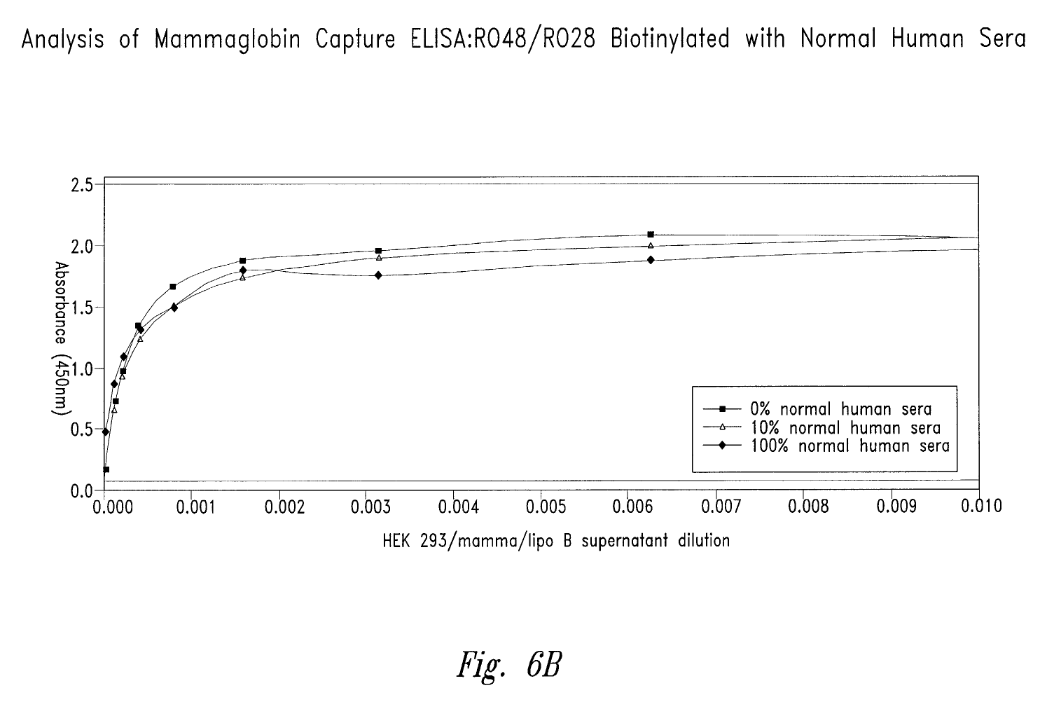

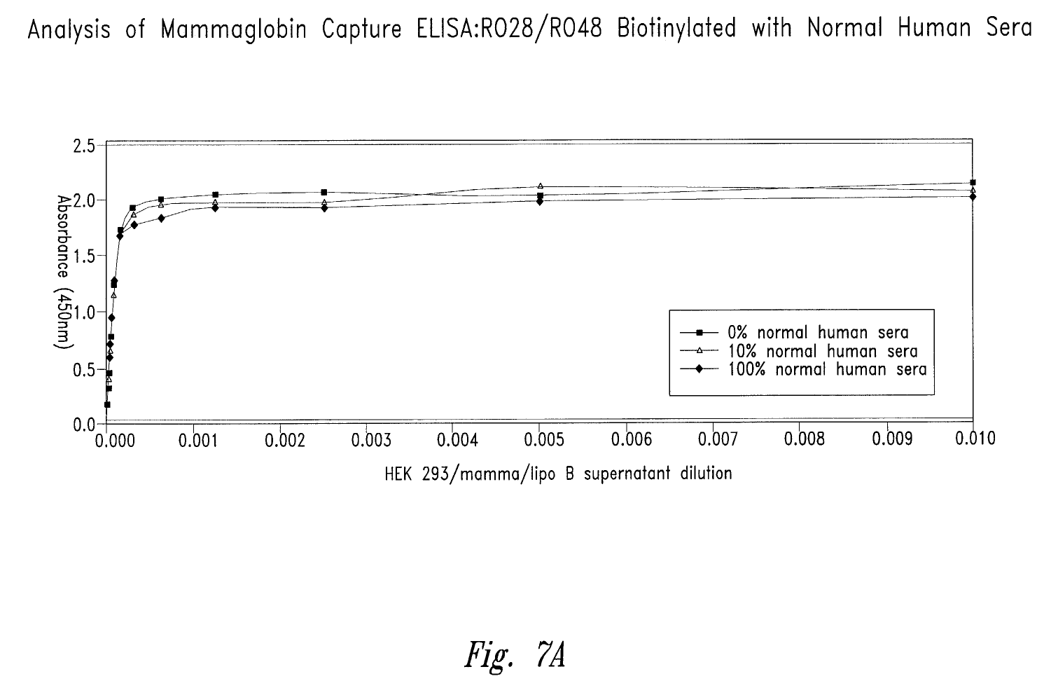

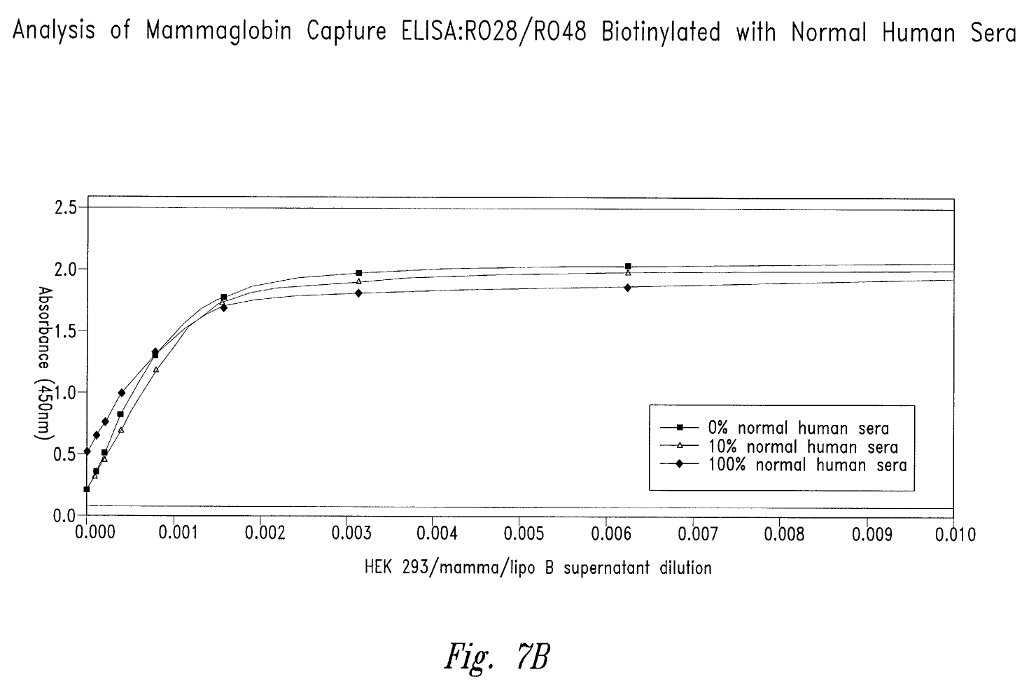

As shown in FIG. 5, FIG. 6, and FIG. 7, the combinations of coating plates with RO28 and detecting with biotinylated RO48 antibody, and coating plates with RO48 and detecting with biotinylated RO28 antibody were the most sensitive. This capture ELISA assay detects mammaglobin associated with lipophilin B. When this assay was used to test sera from patients with breast cancer, higher levels of mammaglobin were detected in patients with stage I, II and metastatic breast cancer compared to patients with other forms of cancer, or with DCIS, LCIS or no cancer.

### Example 10

### Immune Responses to Recombinant Mammaglobin, Native Mammaglobin-Liphilin B Complex, and Lipophilin B in Sera from Pateints with Different Stage Breast Cancer

This example describes the immune responses to a recombinant mammaglobin, native mammaglobin-lipophilin B complex and lipophilin B in sera from patients with breast cancer at different stages of disease as well as in other cancers and healthy donor sera to determine the prognostic utility of such antibodies.

Patient Samples

Sera were obtained from 74 breast cancer patients with various stages of disease progression as well as from 26 ovarian, 30 endometrial, 39 prostate and 30 lung cancer patients (Samplex, Westlake Village, Calif. and Lifeblood, Memphis, Tenn.).

Healthy Donor Sera

Twenty healthy donor sera were obtained from Boston Biomedica Inc (West Bridgewater, Mass.) as well as from volunteer donors at Corixa Corp (Seattle, Wash.).

Peptides

Mature lipophilin B is a 69 amino acid peptide (amino acids 22-90 of the sequence set forth in SEQ ID NO:2) and was synthesized using Fmoc chemistry employing HBTU (2-(1H-benzotriazol-1-yl)-1,1,3,3-tetramethyluronium hexafluro phosphate) on a Pioneer peptide synthesizer (PE Biosystems, Foster City, Calif.). The peptide was cleaved from the solid support using standard procedures and purified by reverse phase high performance liquid chromatography on a C18 column (Vydac), using a gradient of 5-60% acetonitrile (containing 0.05% trifluoroactic acid) in water (containing 0.05% trifluoroacetic acid). The purified peptide was lyophilized and characterized by MALDI mass spectrometry prior to use.

Recombinant Mammaglobin

Recombinant mammaglobin was expressed in E. coli. cDNA constructs of the full-length mature mammaglobin (amino acid sequence set forth in SEQ ID NO:1) with N- and C-terminal His-Tags were subcloned into a modified pET 28 vector using standard techniques. The construct was then transformed into BL21 pLysS E. coli and grown in culture in the presence of kanamycin and chloramphenicol before inducing with iso-propyl-β-D-thiogalactosisde. Recombinant protein was purified using nickel affinity chromatography using an imidazole gradient.

Mammaglobin/Lipophilin B Complex

Native mammaglobin/lipophilin B complex was isolated from supernatants obtained from MDA-MB415 cells grown in serum free media (26).

ELISA

For Detection of Human Lipophilin B Antibody Responses

Ninety-six well microtiter plates (Corning Costar, Cambridge, Mass.) were coated overnight at 4° C. with lipophilin B peptide (1 ug/well), recombinant mammaglobin (200 ng/well) or purified native mammaglobin/lipophilin B complex (500 ng/well)). Plates were then aspirated and blocked with phosphate buffered saline (PBS) containing 5% non-fat milk for 2 hours at room temperature. This was followed by washing in PBS containing 0.1% Tween 20 (PBST). Serum (1/100) dilution in PBS containing 5% non-fat dried milk was added to wells and incubated for 2 hours at room temperature. This was followed by washing 6 times with PBST, and then incubating with protein A-HRP conjugate at a 1/20000 dilution in PBST with 0.1% BSA (Sigma Chemical Co, St Louis, Mo.) for 1 hour. Plates were then washed 6 times in PBST and then incubated with Tetramethylbenzidine (TMB) substrate (Kirkegaard and Perry, Gaithersburg Md.) for 15 minutes. The reaction was stopped by the addition of 1 N sulfuric acid and plates read at 450 nm using an ELISA plate reader (Model Elx800, Biotek instruments, Hyland Park Va.). The cut-off for assays was determined from the mean optical density of the negative population plus three standard deviations of the mean. The signal to cut-off was determined from the ratio of the sample ELISA optical density value to the cut-off. In assays to determine titer serum was used at 1:50-1:6400 dilution in the assay.

Antibodies

A mouse (2D3) monoclonal antibody was made to recombinant mammaglobin using conventional methods. A rabbit polyclonal was prepared to lipophilin B as well as the mammaglobin-lipophilin B complex isolated from MB415 cell line supernatants.

Results

Lipophilin B Specific Antibodies in Sera of Breast Cancer Patients:

Studies were performed to determine the presence of antibodies to recombinant mammaglobin, the native complex of mammaglobin and lipophilin B and free lipophilin B peptide in the sera of breast cancer patients. Differential responses were observed in all three cases. Antibodies to the lipophilin B synthetic peptide were observed in 20/74 sera from breast cancer patients. Of the stage 1V breast cancer sera tested, 13/35 were positive for antibodies to lipophilin B as shown in Table 1 with indications of increased antibody titer in later stage tumors. The presence of lower titer antibodies in earlier stage tumors is also shown in Table 1. In contrast, weak immune responses were seen in some sera from breast cancer patients with mammaglobin and native complex, but in different sera than those reactive to the synthetic lipophilin B peptide (Table 2).

TABLE 1
Antibody responses to lipophilin B in breast, ovarian, lung
and prostate tumors

| | | Signal/cut-off Mean ± Std Dev |
|---|---|---|
| Sample ID | Number positive | (95% confidence Interval) |
| Breast Tumor | 20/74 | 2.59 ± 6.54 (1.10-4.08) |
| Total | | |
| Stage I | 2/10 | 0.77 ± 0.71 (0.33-1.21) |
| Stage II | 5/21 | 0.83 ± 0.90 (0.45-1.22) |
| Stage III | 0/8 | 0.29 ± 0.17 (0.18-0.41) |
| Stage IV | 13/35 | 4.68 ± 9.08 (1.67-7.69) |
| Ovarian Tumor | 6/26 | 0.91 ± 0.70 (0.64-1.18) |
| Endometrial | 1/30 | 0.63 ± 0.16 (0.57-0.69) |
| tumor | | |
| Prostate Tumor | 3/39 | 0.51 ± 0.34 (0.40-0.61) |
| Lung Tumor | 1/30 | 0.34 ± 0.21 (0.26-0.41) |
| Healthy donors | 0/20 | 0.36 ± 0.21 (0.27-0.45) |

TABLE 2
Breast cancer sera and antibody responses to recombinant
mammaglobin and native mammaglobin complex
Target Antigen

| Sample ID | Mammaglobin | Native Complex |
|---|---|---|
| Breast Tumor: | | |
| Stage I | | 1/10 |
| Stage II | | 2/21 |
| Stage III | | 0/8 |
| Stage IV | | 8/35 |
| Healthy donors | 0/20 | 0/20 |

Higher titer antibodies to lipophilin B were shown to be more prevalent in late stage tumors with 13/35 stage 1V tumors exhibiting antibodies, some of which had titers of >1:1000. Titration curves showed an increase in titer in sera from stage 1V patients (n=13) as compared to earlier stages of breast cancer (n=7) and healthy donors (n=7).

Lipophilin B Antibodies in Other Cancers and Normal Donors

Sera from patients with ovarian, prostate, and lung cancer were also tested for the presence of lipophilin B antibodies. Low titer antibodies were seen in sera from 6/26, 1/30, and 3/39 ovarian, endometrial and prostate cancer patients, respectively. Of 30 lung cancer patient sera tested only one showed a borderline response (signal/cut-off 1.23). The mean signal to cut-off values for the 6 positive ovarian tumors was 0.91 that compares to 2.59 for all breast cancer sera and 4.68 for Stage 1V breast cancer.

No antibody response to lipophilin B alone, complexed with mammaglobin or to free recombinant mammaglobin was observed in 20 normal donor sera further supporting the specific response to lipophilin B observed in breast cancer patients. While there were detectable antibody levels in several sera from ovarian cancer patients the levels were significantly less than seen in breast cancer patients. Prostate and lung cancer sera had little or no significant antibody responses to lipophilin B (See Table 1). The detection of low responses to ovarian cancers is consistent with the 10 fold lower mRNA expression levels detectable in ovarian tumors than in breast tumors.

Antibody Responses to Mammaglobin, Complex and Lipophilin B

The human antibody response to the mammaglobin-lipophilin B complex or recombinant mammaglobin was also frequently different to that seen with lipophilin B. A monoclonal antibody 2D3 raised to the recombinant only reacted with the recombinant and not to the native complex or lipophilin B. In contrast, the polyclonal anti complex antibody reacted with both the recombinant and the complex but not lipophilin B. The polyclonal antibody to lipophilin B reacted with the complex as well as lipophilin B but not recombinant mammaglobin. In human breast cancer sera very little antibody response was seen to recombinant mammaglobin. The strongest responses were to lipophilin B, but also to the complex. However, in many cases the sera reactive with the complex were not the same as those reacting to lipophilin B implying different epitopes are involved.

ELISA detected antibodies specific to lipophilin B in 27% (20/74) sera from breast cancer patients. Higher titer antibodies, with in some cases titers >1:1000, were observed in stage IV tumors (37.1%, 13/35). In contrast, only weak responses were observed to mammaglobin, either as a purified recombinant or complexed with lipophilin B. The strong response to lipophilin B and not to the complex would also indicate that lipophilin B may exist in the sera of breast cancer patients in the free form. It may also indicate that in the complex it is folded so that it is not accessible. Sera that were positive for lipophilin B antibodies should also be different from those expected to exhibit responses to HER-2/neu which tend to predominate in early stage tumors (Disis, M. L., Pupa, S. M., Gralow, J. R., Dittadi, R., Menard, S., and Cheever, M. A. High titer HER-2/neu protein specific antibody can be detected in patients with early stage breast cancer. J. Clin Oncol, 15(11): 3363-7, 1997.). While some sera were reactive with the native mammaglobin-lipophilin B complex, they tended to be different from those with lipophilin B antibodies indicating the involvement of different epitopes.

In summary, there is a high degree of correlation between the antibodies to lipophilin B in serum to the presence of breast cancer. Similar antibody responses have been seen to other breast cancer markers e.g. HER-2/neu. The presence of antibodies to HER-2/neu has been highly indicative of the potential of this protein as a target for immunotherapy and indeed it has been targeted for monoclonal antibody therapy and as a vaccine target. Many tumors however, do not express HER-2/neu and there is a need to identify other candidate antigens that exhibit humoral responses in cancer patients that may be immunotherapeutic targets. For this purpose SEREX techniques have been used to identify such antigens. Lipophilin B, while eliciting an immune response, is also linked to mammaglobin a highly glycosylated protein that may complicate its use in immunotherapy. The presence of lipophilin B specific antibodies in serum may, however, serve as a diagnostic indicator of breast cancer.

### Example 11

### Analysis of Lipophilin B cDNA Expression Using Real-Time PCR

Breast tumors, ovarian tumors and prostate tumors along with their corresponding normal tissue and other normal tissues were tested in quantitative (real-time) PCR. The first-strand cDNA used in the quantitative real-time PCR was synthesized from 20 μg of total RNA that was treated with DNase I (Amplification Grade, Gibco BRL Life Technology, Gaithersburg, Md.), using Superscript Reverse Transcriptase (RT) (Gibco BRL Life Technology, Gaithersburg, Md.). Real-time PCR was performed with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence was monitored during the whole amplification process. The primers used for lipophilin B detection were: Forward 5′-TGCCCCTCCGGAAGCT-3′ (SEQ ID NO:77) and reverse: 5′-CGTTTCTGMGGGACATCTGATC-3′ (SEQ ID NO:78). The optimal concentration of primers was determined using a checkerboard approach and a pool of cDNAs from tumors was used in this process. The PCR reaction was performed in 25 μl volumes that included 2.5 μl of SYBR green buffer, 2 μl of cDNA template and 2.5 μl each of the forward and reverse primers for the gene of interest. The cDNAs used for RT reactions were diluted 1:10 for each gene of interest and 1:100 for the β-actin control. In order to quantitate the amount of specific cDNA (and hence initial mRNA) in the sample, a standard curve was generated for each run using the plasmid DNA containing the gene of interest. Standard curves were generated using the Ct values determined in the real-time PCR which were related to the initial cDNA concentration used in the assay. Standard dilution ranging from 20-2×10⁶ copies of the gene of interest was used for this purpose. In addition, a standard curve was generated for β-actin ranging from 200 fg-2000 pg. This enabled standardization of the initial RNA content of a tissue sample to the amount of β-actin for comparison purposes. The mean copy number for each group of tissues tested was normalized to a constant amount of β-actin, allowing the evaluation of the over-expression levels seen with each of the genes.

As is shown in Table 3, Lipophilin B is over-expressed in breast tumors as compared to normal breast and a panel of other normal tissues. Lipophilin B exhibits an mRNA expression profile in breast tumors similar to mammaglobin, and while both share some mRNA expression in skin and salivary gland, lipophilin B is also expressed in skeletal muscle, adrenal gland, cartilage and retina (Table 3). However, unlike mammaglobin that is specific for breast tumors, lipophilin B also is expressed at ˜10 fold lower levels in ovarian cancers and prostate cancers. No expression of lipophilin B was detected in colon tumors (Table 3). Both lipophilin B and mammaglobin had similar mRNA expression profiles in a tumor cell line panel comprised of breast, prostate, ovarian and colon cell lines. On this panel, both lipophilin B and mammaglobin had mRNA levels elevated in MDA-MB415 (+++++), BT474 (++++) and SKBR-3 (+) cells with relatively similar expression profiles. Lipophilin B mRNA was expressed at a low level in LNCaP prostate cancer derived cells.

TABLE 3
Lipophilin B mRNA expression in normal
and cancer tissues determined by Quantitative PCR

| Tissue | N | Mean copies/ng B-actin | Positive |
|---|---|---|---|
| Breast Tumor | 25 | 3393.41 ± 12724.51 | 10 |
| Breast Normal | 4 | 175.07 ± 197.44 | 2 |
| Ovarian Tumor | 22 | 275.14 ± 457.54 | 12 |
| Ovarian Normal | 5 | 27.06 ± 33.03 | 0 |
| Prostate Tumor | 24 | 98.52 ± 126.85 | 7 |
| Prostate Normal | 4 | 87.16 ± 63.27 | 1 |
| Colon Tumor | 26 | 0.37 ± 0.71 | 0 |
| Colon Normal | 7 | 0.89 ± 0.79 | 0 |
| Skin | 4 | 2199.53 ± 2565.88 | 3 |
| Skeletal Muscle | 3 | 686.71 ± 913.27 | 2 |
| Salivary gland | 2 | 2497.48 ± 3493.77 | 1 |
| Adrenal | 2 | 129.82 ± 161.34 | 1 |
| Retina | 2 | 120.14 ± 114.49 | 1 |
| All Other tissues | 60 | 14.25 ± 23.16 | 2 |

Cut-off = Mean normals + 3SD = 83.7

### Example 12

### Analysis of Lipophilin B Polymorphism

Because of the disulfide linkages between mammaglobin and lipophilin B the analysis of cDNA polymorphisms in lipophilin B was studied to determine their potential impact on antibody responses in breast cancer patients.

The polymorphism study focused primarily on cDNA from primary and metastatic breast tumors. Briefly, the approach used was to create single stranded cDNA from total RNA isolated from each tumor. This was used as a template for PCR amplification of lipophilin B genes. The tumors used in the analysis had been previously shown to express lipophilin B mRNA by quantitative PCR. Lipophilin B primers were selected outside of the region encoding the ORF so that sequence variants within and adjacent to the coding region could be detected. Primers were designed outside the region encoding the ORF excepting that the 5′ primer included part of the region encoding the signal sequence, due to the constraints of the 5′ UTR sequence on primer design. This enabled sequence variants within and adjacent to the coding region to be detected. Each amplification of lipophilin B was performed with Pfu polymerase to limit to a minimum the introduction of PCR induced sequence variants. Each of the PCR amplifications was sub-cloned, and five independent clones were subjected to DNA sequence analysis. Both strands were sequenced to reduce the possibility of sequencing errors.

Twenty breast tumors (11 metastatic, 9 primary) were used in this study, along with a single normal breast sample. The analysis of the variants revealed one prevalant variant the sequence of which comprised a C-T transition at base pair 158 of wild type Lipophilin B (SEQ ID NO:10) that resulted in a proline to leucine change at the amino acid level. The cDNA and amino acid sequence for this variant are set forth in SEQ ID NOs:12 and 40, respectively. Eleven of the twenty tumors analyzed contained cDNAs with this sequence variant.

### Example 13

### Epitope Mapping of Lipophilin B Relative to Known Polymorphisms

Several overlapping peptides were synthesized that spanned the full-length lipophilin B sequence (see Table 4, amino acid sequences set forth in SEQ ID NOs:68-76). This included peptides that represented the proline to leucine change discussed in Example 12. All of these peptides were tested in ELISA with a rabbit polyclonal antisera made to the proline version and to human breast cancer sera to fine map the epitopes. Rabbit anti-lipophilin B reacted specifically with the C terminal peptide 7 (SEQ ID NO:76) but was also reactive with peptides in and around the region where the proline to leucine polymorphism occurs, with peptides 3A and B being reactive as well as 4A (SEQ ID NOs:70, 71, and 72, respectively). Human sera reactivity, however, appeared to be located in peptide 5, but with less overall reactivity than the full-length lipophilin B peptide. Such a response should therefore be largely independent of sequence variation occuring at upstream sites (peptides 3A and B).

TABLE 4

Overlapping Peptides Used to Map Lipophilin
Antibody Epitopes

| | | |
|---|---|---|
| Peptide | Peptide ID | SEQ ID NO: |

| | | |
|---|---|---|
| EFCPALVSELLDFFF | 1 | 68 |
| LLDFFFISEPLFKLS | 2 | 69 |
| PLFKLSLAKFDAPPE | 3A | 70 |
| PLFKLSLAKFDAPLE | 3B | 71 |
| FDAPPEAVAAKLGVK | 4A | 72 |
| FDAPLEAVAAKLGVK | 4B | 73 |
| AKLGVKRCTDQMSLQ | 5 | 74 |
| DQMSLQKRSLIAEVL | 6 | 75 |
| LIAEVLVKILKKCSV | 7 | 76 |

Epitope mapping of the polymorphs of lipophilin B indicated that the predominant human antibody response was to peptide 5. This is in contrast to the location of epitopes reacting with a rabbit polyclonal antibody to lipophilin B. peptides 3Pro, 3Leu, 4Pro and 7. Reactivity to peptide 5, however, is much weaker than seen with the complete lipophilin B protein indicating the possibility of conformational epitopes contributing to the recognition by human sera.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. A fusion protein, comprising a first lipophilin-like polypeptide linked by a peptide bond to a second lipophilin-like polypeptide.

2. The fusion protein according to claim 1 wherein said first lipophilin-like polypeptide comprises a mammaglobin polypeptide and said second lipophilin-like polypeptide comprises a lipophilin polypeptide.

3. A polynucleotide encoding the fusion protein according to claim 1.

4. An expression vector, comprising the polynucleotide of claim 3 operably linked to an expression control sequence.

5. A host cell transformed or transfected with the expression vector according to claim 4.

6. An isolated antibody, or antigen-binding fragment thereof, that specifically binds to the polypeptide of claim 1 wherein said antibody or fragment does not detectably bind to mammaglobin, lipophilin A, lipophilin B, or lipophilin C.

7. The antibody according to claim 6 wherein said antibody is a monoclonal antibody.

8. A pharmaceutical composition, comprising the antibody, or antigen binding fragment thereof according to claim 6, in combination with a physiologically acceptable carrier.

9. A method for determining the presence or absence of a cancer in a patient, comprising the steps of:
(a) contacting a biological sample obtained from a patient with the antibody or antigen-binding fragment thereof according to claim 6.
(b) detecting in said sample an amount of complex that binds to said antibody or fragment thereof; and
(c) comparing the amount of complex detected with a predetermined cut-off value,
wherein an elevated amount of complex relative to said predetermined cut-off value indicates the presence or absence of a cancer in said patient.

10. The method according to claim 9 wherein said antibody is a monoclonal antibody.

11. The method according to claim 9 wherein step (b) comprises a two-antibody sandwich assay.

12. The method according to claim 9 wherein step (b) comprises contacting bound complex with a second antibody that specifically binds to a mammaglobin or lipophilin B epitope.

13. The method according to claim 9, wherein said antibody or fragment thereof specifically binds lipophilin B, and wherein step (b) comprises contacting bound complex with a second antibody, or antigen-binding fragment thereof, that specifically binds glycosylated mammaglobin.

14. The method according to claim 9, wherein said cancer is breast, ovarian or prostate cancer.

15. A diagnostic kit, comprising:
(a) one or more antibodies or antigen-binding fragments thereof according to claim 6; and
(b) a detection reagent comprising a reporter group.

16. The kit according to claim 15, wherein at least one of said antibodies or fragments thereof is immobilized on a solid support.

17. The kit according to claim 16, wherein said solid support comprises nitrocellulose, latex or a plastic material.

18. The kit according to claim 15, wherein the detection reagent comprises an immunoglobulin, anti-immunoglobulin, protein G, protein A or lectin.

19. The kit according to claim 15, wherein said reporter group is selected from the group consisting of radioactive groups, fluorescent groups, luminescent groups, enzymes, biotin and dyes.

20. A diagnostic kit, comprising:
(a) one or more fusion proteins according to claim 1; and
(b) a detection reagent comprising a reporter group.

21. The kit according to claim 20, wherein said fusion protein(s) are immobilized on a solid support.

22. The kit according to claim 21, wherein said solid support comprises nitrocellulose, latex or a plastic material.

23. The kit according to claim 20, wherein said detection reagent comprises an immunoglobulin, anti-immunoglobulin, protein G, protein A or lectin.

24. The kit according to claim 20, wherein said reporter group is selected from the group consisting of radioactive groups, fluorescent groups, luminescent groups, enzymes, biotin and dyes.

25. A fusion protein, comprising a lipophilin-like polypeptide linked by a peptide bond to a breast tumor antigen.

26. The fusion protein according to claim 25, wherein said breast tumor antigen comprises B305D.

27. The fusion protein according to claim 25, wherein said lipophilin-like polypeptide comprises a mammaglobin polypeptide.

28. The fusion protein according to claim 25, wherein said lipophilin-like polypeptide comprises a lipophilin polypeptide.

29. An isolated antibody, or antigen-binding fragment thereof, that specifically binds to the fusion protein of claim 25, wherein said antibody or fragment does not detectably bind to mammaglobin, lipophilin A, lipophilin B, lipophilin C, or B305D.

30. The antibody according to claim 29, wherein said antibody is a monoclonal antibody.

31. A polynucleotide encoding the fusion protein according to claim 25.

32. An expression vector comprising the polynucleotide of claim 31 operably linked to an expression control sequence.

33. A host cell transformed or transfected with the expression vector according to claim 32.

34. A composition comprising a first component selected from the group consisting of physiologically acceptable carriers and immunostimulants, and a second component selected from the group consisting of:
(a) the fusion protein according to any one of claims 1, 2, and 25; and
(b) the polynucleotide according to claim 3 or 31;

35. A method for stimulating an immune response in a patient, comprising administering to the patient the composition of claim 34.

36. A method for inhibiting the development of a cancer in a patient, comprising administering to the patient the composition of claim 34.

37. The method according to claim 36, wherein said patient is afflicted with breast, ovarian, or prostate cancer.

38. The method according to claim 36, wherein said composition is administered prophylactically to a patient not detectably afflicted with a cancer.

39. An isolated antibody, or antigen-binding fragment thereof, that specifically binds to lipophilin B.

40. The antibody according to claim 39, wherein said antibody is a monoclonal antibody.

41. A pharmaceutical composition comprising the antibody, or antigen binding fragment thereof, according to claim 39, in combination with a physiologically acceptable carrier.

42. An isolated antibody, or antigen-binding fragment thereof, that specifically binds to a lipophilin complex.

43. The antibody according to claim 42 wherein said antibody or fragment does not detectably bind to mammaglobin, lipophilin A, lipophilin B, or lipophilin C.

44. The antibody according to claim 42 wherein said antibody is a monoclonal antibody.

45. A pharmaceutical composition, comprising the antibody, or antigen binding fragment thereof according to claim 42, in combination with a physiologically acceptable carrier.

46. A method for determining the presence or absence of a cancer in a patient, comprising the steps of:
(a) contacting a biological sample obtained from a patient with the antibody or antigen-binding fragment thereof according to claim 42.
(b) detecting in said sample an amount of complex that binds to said antibody or fragment thereof; and
(c) comparing the amount of complex detected with a predetermined cut-off value,
wherein an elevated amount of complex relative to said predetermined cut-off value indicates the presence or absence of a cancer in said patient.

47. The method according to claim 46 wherein said antibody is a monoclonal antibody.

48. The method according to claim 46 wherein step (b) comprises a two-antibody sandwich assay.

49. The method according to claim 46 wherein step (b) comprises contacting bound complex with a second antibody that specifically binds to a mammaglobin or lipophilin B epitope.

50. The method according to claim 46, wherein said antibody or fragment thereof specifically binds lipophilin B, and wherein step (b) comprises contacting bound complex with a second antibody, or antigen-binding fragment thereof, that specifically binds glycosylated mammaglobin.

51. The method according to claim 46, wherein said cancer is breast, ovarian or prostate cancer.

52. A diagnostic kit, comprising:
(a) one or more antibodies or antigen-binding fragments thereof according to claim 42; and
(b) a detection reagent comprising a reporter group.

53. The kit according to claim 52, wherein at least one of said antibodies or fragments thereof is immobilized on a solid support.

54. The kit according to claim 52, wherein said solid support comprises nitrocellulose, latex or a plastic material.

55. The kit according to claim 52, wherein the detection reagent comprises an immunoglobulin, anti-immunoglobulin, protein G, protein A or lectin.

56. The kit according to claim 52, wherein said reporter group is selected from the group consisting of radioactive groups, fluorescent groups, luminescent groups, enzymes, biotin and dyes.

57. An isolated antibody, or antigen-binding fragment thereof, that specifically binds to a lipophilin B polypeptide wherein the lipophilin B polypeptide comprises an amino acid sequence of any one of SEQ ID NOs:68-76.
